(19) United States
(12) Patent Application Publication
(10) Pub. No.: US 2019/0111016 A1
(43) Pub. Date: Apr. 18, 2019

(54) METHODS OF TREATING MUSCLE AND LIVER DISORDERS

(71) Applicant: The Regents of the University of California, Oakland, CA (US)

(72) Inventors: Gino Cortopassi, Davis, CA (US); Genki Hayashi, San Diego, CA (US)

(73) Assignee: The Regents of the University of California, Oakland, CA (US)

(21) Appl. No.: 16/110,719

(22) Filed: Aug. 23, 2018

Related U.S. Application Data
(63) Continuation of application No. PCT/US2017/019474, filed on Feb. 24, 2017.
(60) Provisional application No. 62/300,493, filed on Feb. 26, 2016.

(51) Int. Cl. A61K 31/225; A61K 9/00; A61K 9/51; A61K 47/40

(52) U.S. Cl. CPC A61K 31/225; A61K 47/40; A61K 9/51; A61K 9/0019

(57) ABSTRACT

Provided are methods of treating muscle and liver disorders, and for increasing mitochondrial mass and/or functionality in a mammalian myocyte and/or hepatocyte.

### CROSS-REFERENCE TO RELATED APPLICATIONS

The present application is a continuation of International Patent Application No. PCT/US2017/019474, filed Feb. 24, 2017, which claims priority to U.S. Provisional Application No. 62/300,493, filed on Feb. 26, 2016, the disclosures of which are herein incorporated by reference in their entirety for all purposes.

### BACKGROUND

Inheritance of defects in mitochondrial genes causes mitochondrial disease (1); and at the current time there is no effective or approved therapy for mitochondrial disease. One therapeutic strategy for mitochondrial disease is to increase mitochondrial biogenesis, the idea being that a small defect in function might be ameliorated by increased mitochondrial mass or function overall (2).

The co-transcriptional regulation factor peroxisome proliferator-activated receptor gamma coactivator 1-alpha (PGC1α) is a well-known marker of mitochondrial biogenesis (3). PGC1α induces the expression of the transcription factors, nuclear respiration factor 1 (NRF1) (4). NRF1 was initially identified to regulate nuclear-encoded mitochondrial complex expression (5). However, it has more recently been observed to be involved in mitochondrial replication and even drive the expression of mitochondrially encoded genes (6, 7). Together, PGC1α and NRF1 mediate the expression of mitochondrial transcription factor A (TFAM), a major regulator of mitochondrial replication and transcription (8, 9). Also, expression of TFAM has been shown to be proportional to alterations in mtDNA copy number (10). Thus, TFAM and NRF1 are robust markers of mitochondrial proliferation.

Dimethyl fumarate (DMF) is known for its anti-inflammatory and cytoprotective properties (11, 12). It is currently used to treat multiple sclerosis (MS) and psoriasis and is marketed under the name Tecfidera (13) and Fumaderm (14), respectively. DMF is known to stimulate the activity of the transcription factor, nuclear factor (erythroid-derived 2)-like 2 (Nrf2, also known as NFE2L2) and the G protein coupled receptor, hydroxycarboxylic acid receptor 2 (HCAR2) (15)

Nrf2 helps to maintain cellular redox homeostasis by regulating a number of genes involved in antioxidant protection including, but not limited to, glutathione (16, 17), thioredoxin (18), heme oxygenase (HO1), and NAD(P)H dehydrogenase (NQO1) (19, 20). It was previously discovered that monomethyl fumarate (MMF), a metabolite of DMF, mediates Nrf2 activation by modifying numerous cysteine (Cys) residues of the Kelch-like ECH-associated protein 1 (KEAP1). The modification of KEAP1 then drives the dissociation and translocation of Nrf2 into the nucleus, initiating the transcription of many phase II antioxidant enzymes that contain the antioxidant response element (ARE) promoter sequence (21-23). It is known that knocking out Nrf2 is detrimental to mitochondrial health, and activation of the Nrf2 pathway by DMF is thought to be beneficial to mitochondria by mitigating reactive oxygen species (ROS)-related damage (24).

In addition, Nrf2 is also thought to be involved in the induction of mitochondrial biogenesis. Specifically, Nrf2 is known to positively regulate NRF1 by binding to the four ARE promoter sequences of NRF1, leading to the activation of NRF1 mediated mitochondrial biogenesis pathway (25). In concurrence, a study by Shen et al. 2008 has shown that treatment of murine 3T3-L1 adipocytes with (R)-α-lipoic acid and acetyl-L-carnitine, known activators of Nrf2 induces mitochondrial proliferation and observed increased mtDNA, mitochondrial complex expression, oxygen consumption, and increased expressions of mitochondrial biogenesis biomarkers such as PGC1α, TFAM and NRF1 (26).

HCAR2 is involved in the regulation of anti-inflammatory activity and fat metabolism. DMF's major metabolite MMF is known to be a potent agonist of HCAR2 (27). The effects of DMF on HCAR2 remain largely unclear. However, DMF's protective effect in MS may include its metabolism to MMF that agonizes HCAR2 to cause anti-inflammatory activity in the mouse EAE model of MS (15).

### SUMMARY

In one aspect, provided are methods for promoting and/or increasing mitochondrial mass and/or functionality (e.g., oxygen consumption rate) in a mammalian myocyte and/or hepatocyte. In some embodiments, the methods comprise contacting the myocyte and/or hepatocyte with a compound of Formula (I) or a pharmaceutically acceptable salt thereof; wherein R¹ and R² are independently selected from —CH₃, —OH, —O, -E, and C1-C8 alkoxy (branched or unbranched), provided that at least one of R¹ and R² is C1-C8 alkoxy:

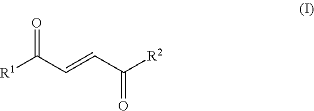

under conditions sufficient to increase mitochondrial mass and/or functionality (e.g., oxygen consumption rate) in a mammalian myocyte and/or hepatocyte. In varying embodiments, the compound of Formula (I) comprises a fumarate ester. In varying embodiments, the compound of Formula (I) is selected from the group consisting of monomethyl fumarate (MMF), monomethyl maleate, monoethyl fumarate, monoethyl maleate, monobutyl fumarate, monobutyl maleate, monooctyl fumarate, monoctyl maleate, mono (phenylmethyl) fumarate, mono (phenylmethyl) maleate, mono (2-hydroxypropyl) fumarate, mono (2-hydroxypropyl) maleate, mono (2-ethylhexyl) fumarate, mono (2-ethylhexyl) maleate, dimethylfumarate, dimethyl maleate, diethyl fumarate, diethyl maleate, dipropyl fumarate, dipropyl maleate, diisopropyl fumarate, diisopropyl maleate, dibutyl fumarate, dibutyl maleate, diisobutyl fumarate, diisobutyl maleate, diheptyl fumarate, diheptyl maleate, bis (2-ethylhexyl) fumarate, bis (2-ethylhexyl) maleate, (−)-Dimenthyl fumarate, (−)-Bis ((S)-1-(ethoxycarbonyl)ethyl) fumarate, (−)-Bis ((S)-1-(ethoxycarbonyl)ethyl) maleate, Bis (2-trifluoroethyl) fumarate, Bis (2-trifluoroethyl) maleate, and mixtures thereof. In varying embodiments, the compound of Formula (I) comprises dimethyl fumarate (DMF). In varying embodiments, the methods further comprise contacting the myocyte and/or hepatocyte with methylene blue. In some embodiments, the methods comprise contacting the myocyte and/or hepatocyte with methylene blue under conditions sufficient to increase mitochondrial mass and/or functionality (e.g., oxygen consumption rate) in a mammalian myocyte and/or hepatocyte. In varying embodiments, the mitochondrial mass is increased by at least about 25%, e.g., by at least about 30%, 35%, 40%, 45%, 50%, 55%, 60%, 70%, 75%, 80%, 85%, 90%, 95% to about 100%. In varying embodiments, the mitochondrial copy number/nucleus is increased by at least about 25%, e.g., by at least about 30%, 35%, 40%, 45%, 50%, 55%, 60%, 70%, 75%, 80%, 85%, 90%, 95% to about 100%. In varying embodiments, the myocyte and/or hepatocyte is contacted with the compound of Formula (I) and/or methylene blue at a concentration in the range of about 1 μM to about 50 μM, e.g., at a concentration in the range of about 1 μM to about 30 μM. In varying embodiments, the compound of Formula (I) and/or methylene blue is formulated in in a cyclodextrin. In varying embodiments, the cyclodextrin is selected from the group consisting of hydroxypropyl-β-cyclodextrin, endotoxin controlled β-cyclodextrin sulfobutyl ethers, or cyclodextrin sodium salts. In varying embodiments, the myocyte and/or hepatocyte is human. In varying embodiments, the myocyte and/or hepatocyte is in vitro. In varying embodiments, the myocyte and/or hepatocyte is in vivo. In varying embodiments, the myocyte is a skeletal myocyte or a cardiomyocyte. In varying embodiments, the myocyte is in or from a subject suffering from a muscle disorder. In varying embodiments, the muscle disorder involves muscle wasting. In varying embodiments, the muscle disorder is selected from the group consisting of Cancer cachexia, age-related muscle wasting (sarcopenia), Mitochondrial myopathy, Acid Maltase Deficiency (AMD), Amyotrophic Lateral Sclerosis (ALS), Amyotrophy, Andersen-Tawil Syndrome, Anterior compartment syndrome of the lower leg, Becker Muscular Dystrophy (BMD), Becker Myotonia Congenita, Bethlem Myopathy, Bimagrumab, Bulbospinal Muscular Atrophy (Spinal-Bulbar Muscular Atrophy), Camitine Deficiency, Camitine Palmityl Transferase Deficiency (CPT Deficiency), Cataplexy, Central core disease of muscle, Centronuclear Myopathy, Charcot-Marie-Tooth Disease (CMT), Charley horse, Chronic fatigue syndrome, Chronic progressive external ophthalmoplegia, Congenital Muscular Dystrophy (CMD), Congenital Myasthenic Syndromes (CMS), Congenital Myotonic Dystrophy, Contracture, Cori Disease (Debrancher Enzyme Deficiency), Cramp, Cricopharyngeal spasm, Debrancher Enzyme Deficiency, Dejerine-Sottas Disease (DSD), Dermatomyositis (DM), Diastasis recti, Distal Muscular Dystrophy (DD), Distal spinal muscular atrophy type 2, Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Exercise therapy for idiopathic inflammatory myopathies, Exercise-associated muscle cramps, Exertional rhabdomyolysis, Facioscapulohumeral Muscular Dystrophy (FSH or FSHD), Fibrodysplasia ossificans progressive, Finnish (Tibial) Distal Myopathy, Forbes Disease (Debrancher Enzyme Deficiency), Fukuyama Congenital Muscular Dystrophy, Glycogen storage disease type XI, Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery-Dreifuss Muscular Dystrophy), Hereditary inclusion body myopathy and myositis, Hereditary Motor and Sensory Neuropathy (Charcot-Marie-Tooth Disease), Hyperthyroid Myopathy, Hypertonia, Hypothyroid Myopathy, Inclusion-Body Myositis (IBM) and myopathy, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Laminopathy, Late-onset mitochondrial myopathy, Limb-Girdle Muscular Dystrophy (LGMD), Lou Gehrig's Disease (Amyotrophic Lateral Sclerosis), Macrophagic myofasciitis, McArdle Disease (Phosphorylase Deficiency), Merosin-Deficient Congenital Muscular Dystrophy, Metabolic myopathy, Mitochondrial Myopathy, Miyoshi Distal Myopathy, Motor Neurone Disease, Muscle atrophy, Muscle fatigue, Muscle imbalance, Muscle weakness, Muscle-Eye-Brain Disease, Myasthenia Gravis (MG), Myoadenylate Deaminase Deficiency, Myofibrillar Myopathy, Myopathy, Myopathy, X-linked, with excessive autophagy, Myophosphorylase Deficiency, Myositis, Myositis ossificans, Myostatin-related muscle hypertrophy, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Orofacial myological disorders, Paramyotonia Congenita, Paratonia, Pearson Syndrome, Pelvic floor muscle disorder, Periodic Paralysis, Peroneal Muscular Atrophy (Charcot-Marie-Tooth Disease), Phosphofructokinase Deficiency, Phosphoglycerate Kinase Deficiency, Phosphorylase Deficiency, Polymyositis (PM), Pompe Disease (Acid Maltase Deficiency), Progressive External Ophthalmoplegia (PEO), Psoas muscle abscess, Pyomyositis, Rod Body Disease (Nemaline Myopathy), Sarcoglycanopathy, Sphincter paralysis, Spinal Muscular Atrophy (SMA), Spinal-Bulbar Muscular Atrophy (SBMA)/Kennedy's disease, Steinert Disease (Myotonic Muscular Dystrophy), Strain (injury), Tarui Disease (Phosphofructokinase Deficiency), Thomsen Disease (Myotonia Congenita), Thyrotoxic periodic paralysis, Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), ZASP-Related Myopathy and Zenker's degeneration. In varying embodiments, the muscle disorder is a muscular dystrophy. In varying embodiments, the hepatocyte is in or from a subject suffering from a liver disorder. In varying embodiments, the liver disorder is selected from the group consisting of mitochondrial liver disease, hepatitis, alcoholic liver disease, fatty liver disease (hepatic steatosis), NASH-Non-alcoholic steatohepatitis, Gilbert's syndrome, cirrhosis, primary liver cancer, primary biliary cirrhosis, primary sclerosing cholangitis, and Budd-Chiari syndrome.

In a further aspect, provided are methods of promoting and/or increasing mitochondrial mass and/or functionality (e.g., oxygen consumption rate) in the muscle tissue and/or liver tissue in a subject in need thereof. In another aspect, provided are methods of preventing, delaying, reducing, mitigating, ameliorating and/or inhibiting one or more symptoms associated with a muscle disorder or a liver disorder in a subject in need thereof. In some embodiments, the methods comprise administering to the subject a therapeutically effective regime of a compound of Formula (I) or a pharmaceutically acceptable salt thereof; wherein R1 and R2 are independently selected from —CH₃, —OH, —O, -E, and C1-C8 alkoxy (branched or unbranched), provided that at least one of R¹ and R² is C1-C8 alkoxy:

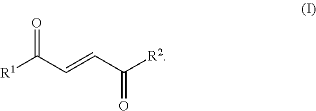

In varying embodiments. the compound of Formula (I) comprises a fumarate ester. In varying embodiments, the compound of Formula (I) is selected from the group consisting of monomethyl fumarate (MMF), monomethyl maleate, monoethyl fumarate, monoethyl maleate, monobutyl fumarate, monobutyl maleate, monooctyl fumarate, monoctyl maleate, mono (phenylmethyl) fumarate, mono (phenylmethyl) maleate, mono (2-hydroxypropyl) fumarate, mono (2-hydroxypropyl) maleate, mono (2-ethylhexyl) fumarate, mono (2-ethylhexyl) maleate, dimethylfumarate, dimethyl maleate, diethyl fumarate, diethyl maleate, dipropyl fumarate, dipropyl maleate, diisopropyl fumarate, diisopropyl maleate, dibutyl fumarate, dibutyl maleate, diisobutyl fumarate, diisobutyl maleate, diheptyl fumarate, diheptyl maleate, bis (2-ethylhexyl) fumarate, bis (2-ethylhexyl) maleate, (−)-Dimenthyl fumarate, (−)-Bis ((S)-1-(ethoxycarbonyl)ethyl) fumarate, (−)-Bis ((S)-1-(ethoxycarbonyl)ethyl) maleate, Bis (2-trifluoroethyl) fumarate, Bis (2-trifluoroethyl) maleate, and mixtures thereof. In varying embodiments, the compound of Formula (I) comprises dimethyl fumarate (DMF). In varying embodiments, the methods further comprise administering to the subject a therapeutically effective regime of methylene blue. In some embodiments, the methods comprise administering to the subject a therapeutically effective regime of methylene blue. In varying embodiments, the compound of Formula (I) and/or methylene blue is administered systemically. In varying embodiments, the compound of Formula (I) and/or methylene blue is administered intravenously. In varying embodiments, the therapeutically effective regime comprises multiple administrations of the compound of Formula (I) and/or methylene blue. In varying embodiments, the therapeutically effective regime comprises administration of the compound of Formula (I) at a dose in the range of from about 200 mg to about 800 mg per day, e.g., in the range of from about 480 mg to about 720 mg per day. In varying embodiments, the therapeutically effective regime comprises administration of methylene blue at a dose in the range of from about 0.25 mg/kg to about 1.0 mg/kg, e.g., from about 0.50 mg/kg to about 1.0 mg/kg, e.g., about 0.25 mg/kg to about 0.50 mg/kg per 1, 2, 3, 4, 5, 6, 7, 8, 9, 10, 11, 12, 13, 14, 15, 16, 17, 18, 19, 20, 21, 22, 23 or 24 hours. In varying embodiments, the therapeutically effective regime comprises administration of methylene blue at a dose in the range of from about 0.25 mg/kg/day to about 1.0 mg/kg/day. In varying embodiments, the compound of Formula (I) and/or methylene blue is formulated as a nanoparticle. In varying embodiments, the compound of Formula (I) and/or methylene blue is formulated for controlled and/or sustained release. In varying embodiments, the compound of Formula (I) and/or methylene blue is formulated in in a cyclodextrin. In varying embodiments, the cyclodextrin is selected from the group consisting of hydroxypropyl-β-cyclodextrin, endotoxin controlled β-cyclodextrin sulfobutyl ethers, or cyclodextrin sodium salts. In varying embodiments, the subject is a human. In varying embodiments, the subject has a muscle disorder or a liver disorder. In varying embodiments, the muscle disorder involves muscle wasting. In varying embodiments, the muscle disorder is selected from the group consisting of Cancer cachexia, age-related muscle wasting (sarcopenia), Mitochondrial myopathy, Acid Maltase Deficiency (AMD), Amyotrophic Lateral Sclerosis (ALS), Amyotrophy, Andersen-Tawil Syndrome, Anterior compartment syndrome of the lower leg, Becker Muscular Dystrophy (BMD), Becker Myotonia Congenita, Bethlem Myopathy, Bimagrumab, Bulbospinal Muscular Atrophy (Spinal-Bulbar Muscular Atrophy), Carnitine Deficiency, Camitine Palmityl Transferase Deficiency (CPT Deficiency), Cataplexy, Central core disease of muscle, Centronuclear Myopathy, Charcot-Marie-Tooth Disease (CMT), Charley horse, Chronic fatigue syndrome, Chronic progressive external ophthalmoplegia, Congenital Muscular Dystrophy (CMD), Congenital Myasthenic Syndromes (CMS), Congenital Myotonic Dystrophy, Contracture, Cori Disease (Debrancher Enzyme Deficiency), Cramp, Cricopharyngeal spasm, Debrancher Enzyme Deficiency, Dejerine-Sottas Disease (DSD), Dermatomyositis (DM), Diastasis recti, Distal Muscular Dystrophy (DD), Distal spinal muscular atrophy type 2, Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Exercise therapy for idiopathic inflammatory myopathies, Exercise-associated muscle cramps, Exertional rhabdomyolysis, Facioscapulohumeral Muscular Dystrophy (FSH or FSHD), Fibrodysplasia ossificans progressive, Finnish (Tibial) Distal Myopathy, Forbes Disease (Debrancher Enzyme Deficiency), Fukuyama Congenital Muscular Dystrophy, Glycogen storage disease type XI, Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery-Dreifuss Muscular Dystrophy), Hereditary inclusion body myopathy and myositis, Hereditary Motor and Sensory Neuropathy (Charcot-Marie-Tooth Disease), Hyperthyroid Myopathy, Hypertonia, Hypothyroid Myopathy, Inclusion-Body Myositis (IBM) and myopathy, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Laminopathy, Late-onset mitochondrial myopathy, Limb-Girdle Muscular Dystrophy (LGMD), Lou Gehrig's Disease (Amyotrophic Lateral Sclerosis), Macrophagic myofasciitis, McArdle Disease (Phosphorylase Deficiency), Merosin-Deficient Congenital Muscular Dystrophy, Metabolic myopathy, Mitochondrial Myopathy, Miyoshi Distal Myopathy, Motor Neurone Disease, Muscle atrophy, Muscle fatigue, Muscle imbalance, Muscle weakness, Muscle-Eye-Brain Disease, Myasthenia Gravis (MG), Myoadenylate Deaminase Deficiency, Myofibrillar Myopathy, Myopathy, Myopathy, X-linked, with excessive autophagy, Myophosphorylase Deficiency, Myositis, Myositis ossificans, Myostatin-related muscle hypertrophy, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Orofacial myological disorders, Paramyotonia Congenita, Paratonia, Pearson Syndrome, Pelvic floor muscle disorder, Periodic Paralysis, Peroneal Muscular Atrophy (Charcot-Marie-Tooth Disease), Phosphofructokinase Deficiency, Phosphoglycerate Kinase Deficiency, Phosphorylase Deficiency, Polymyositis (PM), Pompe Disease (Acid Maltase Deficiency), Progressive External Ophthalmoplegia (PEO), Psoas muscle abscess, Pyomyositis, Rod Body Disease (Nemaline Myopathy), Sarcoglycanopathy, Sphincter paralysis, Spinal Muscular Atrophy (SMA), Spinal-Bulbar Muscular Atrophy (SBMA)/Kennedy's disease, Steinert Disease (Myotonic Muscular Dystrophy), Strain (injury), Tarui Disease (Phosphofructokinase Deficiency), Thomsen Disease (Myotonia Congenita), Thyrotoxic periodic paralysis, Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), ZASP-Related Myopathy and Zenker's degeneration. In varying embodiments, the muscle disorder is a muscular dystrophy, e.g., Becker Muscular Dystrophy (BMD), Congenital Muscular Dystrophy (CMD), Congenital Myotonic Dystrophy, Distal Muscular Dystrophy (DD), Duchenne Muscular Dystrophy (DMD), Dystrophia Myotonica (Myotonic Muscular Dystrophy), Emery-Dreifuss Muscular Dystrophy (EDMD), Facioscapulohumeral Muscular Dystrophy (FSH or FSHD), Fukuyama Congenital Muscular Dystrophy, Hauptmann-Thanheuser MD (Emery-Dreifuss Muscular Dystrophy), Merosin-Deficient Congenital Muscular Dystrophy, Integrin-Deficient Congenital Muscular Dystrophy, Limb-Girdle Muscular Dystrophy (LGMD), Myotonic Muscular Dystrophy (MMD), Oculopharyngeal Muscular Dystrophy (OPMD), Steinert Disease (Myotonic Muscular Dystrophy), Ullrich Congenital Muscular Dystrophy and Walker-Warburg Syndrome (Congenital Muscular Dystrophy). In varying embodiments, the liver disorder is selected from the group consisting of mitochondrial liver disease, hepatitis, alcoholic liver disease, fatty liver disease (hepatic steatosis), NASH-Non-alcoholic steatohepatitis, Gilbert's syndrome, cirrhosis, primary liver cancer, primary biliary cirrhosis, primary sclerosing cholangitis, and Budd-Chiari syndrome. In varying embodiments, the subject does not have a neurodegenerative disorder. In varying embodiments, the subject does not have multiple sclerosis (MS), Alzheimer's disease (AD), amyotrophic lateral sclerosis (ALS), Parkinson's disease (PD), Huntington's disease (HD), Mitochondrial myopathy or a progressive external ophthalmoplegia

### Definitions

As used herein, “administering” refers to local and systemic administration, e.g., including enteral, parenteral, pulmonary, and topical/transdermal administration. Routes of administration for compounds (e.g., compounds of Formula (I), including dimethyl fumarate; methylene blue) that find use in the methods described herein include, e.g., oral (per os (P.O.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “co-administering” or “concurrent administration”, when used, for example with respect to the compounds (e.g., compounds of Formula (I), including dimethyl fumarate; methylene blue) and/or analogs thereof and another active agent (e.g., a cognition enhancer), refers to administration of the compound and/or analogs and the active agent such that both can simultaneously achieve a physiological effect. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the body (e.g., in the plasma) at a significant fraction (e.g., 20% or greater, preferably 30% or 40% or greater, more preferably 50% or 60% or greater, most preferably 70% or 80% or 90% or greater) of their maximum serum concentration for any given dose.

The term “effective amount” or “pharmaceutically effective amount” refer to the amount and/or dosage, and/or dosage regime of one or more compounds necessary to bring about the desired result e.g., increased mitochondria number, increased muscle mass, increased muscle strength, decreased muscle weakness (e.g., therapeutically effective amounts), an amount sufficient to reduce the risk or delaying the onset, and/or reduce the ultimate severity of a disease characterized by amyloid deposits in the brain in a mammal (e.g., prophylactically effective amounts).

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject Such prescribing can include, for example, drafting a prescription form, annotating a medical record, and the like.

The phrase “in conjunction with” when used in reference to the use of the active agent(s) described herein (e.g., compounds of Formula (I), including dimethyl fumarate; methylene blue, or an analogue thereof, an enantiomer, a mixture of enantiomers, a pharmaceutically acceptable salt, solvate, or hydrate of said compound(s) or analogue(s)) in conjunction with one or more other drugs described herein (e.g., an acetylcholinesterase inhibitor) the active agent(s) and the other drug(s) are administered so that there is at least some chronological overlap in their physiological activity on the organism. When they are not administered in conjunction with each other, there is no chronological overlap in physiological activity on the organism. In certain preferred embodiments, the “other drug(s)” are not administered at all (e.g., not co-administered) to the organism.

As used herein, the terms “treating” and “treatment” refer to delaying the onset of, retarding or reversing the progress of, reducing the severity of, or alleviating or preventing either the disease or condition to which the term applies, or one or more symptoms of such disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease. In certain embodiments, the reduction or elimination of one or more symptoms of pathology or disease can include, but is not limited to, muscle wasting, muscle weakness, hepatic dysfunction.

As used herein, the phrase “consisting essentially of” refers to the genera or species of active pharmaceutical agents recited in a method or composition, and further can include other agents that, on their own do not substantial activity for the recited indication or purpose. In some embodiments, the phrase “consisting essentially of” expressly excludes the inclusion of one or more additional agents that have neuropharmacological activity other than the recited compounds (e.g., other than compounds of Formula (I), including dimethyl fumarate; methylene blue). In some embodiments, the phrase “consisting essentially of” expressly excludes the inclusion of one or more additional active agents other than the compounds (e.g., other than compounds of Formula (I), including dimethyl fumarate; methylene blue). In some embodiments, the phrase “consisting essentially of” expressly excludes the inclusion of one or more acetylcholinesterase inhibitors.

The terms “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other healthworker in a hospital, psychiatric care facility, as an outpatient, or other clinical context. In certain embodiments the subject may not be under the care or prescription of a physician or other healthworker.

The symbol “—” means a single bond, “═” means a double bond, “≡” means a triple bond. The symbol “ [symbol] ” refers to a group on a double-bond as occupying either position on the terminus of the double bond to which the symbol is attached; that is, the geometry, E- or Z-, of the double bond is ambiguous and both isomers are meant to be included. When a group is depicted removed from its parent formula, the “ [symbol] ” symbol will [symbol] be used at the end of the bond which was theoretically cleaved in order to separate the group from its parent structural formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual formula as having a hydrogen or hydrogens as substitution (expressly defined hydrogen), for example, CH₂CH₂. It would be understood by one of ordinary skill in the art that the aforementioned descriptive techniques are common in the chemical arts to provide brevity and simplicity to description of otherwise complex structures.

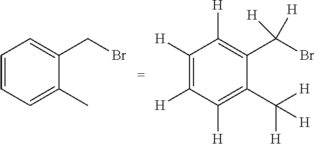

In this application, some ring structures are depicted generically and will be described textually. For example, in the schematic below if ring A is used to describe a phenyl, there are at most four hydrogens on ring A (when R is not H).

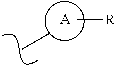

If a group R is depicted as “floating” on a ring system, as for example in the group:

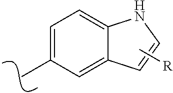

then, unless otherwise defined, a substituent R can reside on any atom of the fused bicyclic ring system, excluding the atom carrying the bond with the “ ” symbol, so long as a stable structure is formed. In the example depicted, the R group can reside on an atom in either the 5-membered or the 6-membered ring of the indolyl ring system.

When there are more than one such depicted “floating” groups, as for example in the formulae:

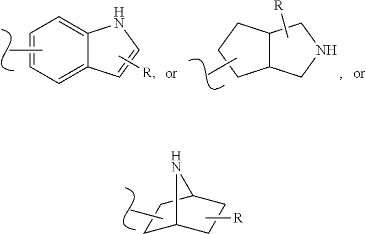

where there are two groups, namely, the R and the bond indicating attachment to a parent structure; then, unless otherwise defined, the “floating” groups can reside on any atoms of the ring system, again assuming each replaces a depicted, implied, or expressly defined hydrogen on the ring system and a chemically stable compound would be formed by such an arrangement.

When a group R is depicted as existing on a ring system containing saturated carbons, as for example in the formula:

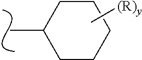

where, in this example, y can be more than one, assuming each replaces a currently depicted, implied, or expressly defined hydrogen on the ring; then, unless otherwise defined, two R's can reside on the same carbon. A simple example is when R is a methyl group; there can exist a geminal dimethyl on a carbon of the depicted ring (an “annular” carbon). In another example, two R's on the same carbon, including that same carbon, can form a ring, thus creating a spirocyclic ring (a “spirocyclyl” group) structure. Using the previous example, where two R's form, e.g. a piperidine ring in a spirocyclic arrangement with the cyclohexane, as for example in the formula:

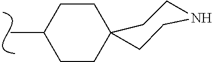

“Alkyl” in its broadest sense is intended to include linear, branched, or cyclic hydrocarbon structures, and combinations thereof. Alkyl groups can be fully saturated or with one or more units of unsaturation, but not aromatic. Generally alkyl groups are defined by a subscript, either a fixed integer or a range of integers. For example, “C₈alkyl” includes n-octyl, iso-octyl, 3-octynyl, cyclohexenylethyl, cyclohexylethyl, and the like; where the subscript “8” designates that all groups defined by this term have a fixed carbon number of eight. In another example, the term “C₁₋₆alkyl” refers to alkyl groups having from one to six carbon atoms and, depending on any unsaturation, branches and/or rings, the requisite number of hydrogens. Examples of C₁₋₆alkyl groups include methyl, ethyl, vinyl, propyl, isopropyl, butyl, s-butyl, t-butyl, isobutyl, isobutenyl, pentyl, pentynyl, hexyl, cyclohexyl, hexenyl, and the like. When an alkyl residue having a specific number of carbons is named generically, all geometric isomers having that number of carbons are intended to be encompassed. For example, either “propyl” or “C₃alkyl” each include n-propyl, c-propyl, propenyl, propynyl, and isopropyl. Cycloalkyl is a subset of alkyl and includes cyclic hydrocarbon groups of from three to thirteen carbon atoms. Examples of cycloalkyl groups include c-propyl, c-butyl, c-pentyl, norbornyl, norbomenyl, c-hexenyl, adamantyl and the like. As mentioned, alkyl refers to alkanyl, alkenyl, and alkynyl residues (and combinations thereof)—it is intended to include, e.g., cyclohexylmethyl, vinyl, allyl, isoprenyl, and the like. An alkyl with a particular number of carbons can be named using a more specific but still generic geometrical constraint, e.g. “C₃₋₆cycloalkyl” which means only cycloalkyls having between 3 and 6 carbons are meant to be included in that particular definition. Unless specified otherwise, alkyl groups, whether alone or part of another group, e.g. —C(O)alkyl, have from one to twenty carbons, that is C₁₋₂₀alkyl. In the example “—C(O)alkyl,” where there were no carbon count limitations defined, the carbonyl of the —C(O)alkyl group is not included in the carbon count, since “alkyl” is designated generically. But where a specific carbon limitation is given, e.g. in the term “optionally substituted C₁₋₂₀alkyl,” where the optional substitution includes “oxo” the carbon of any carbonyls formed by such “oxo” substitution are included in the carbon count since they were part of the original carbon count limitation. However, again referring to “optionally substituted C₁₋₂₀alkyl,” if optional substitution includes carbon-containing groups, e.g. CH₂CO₂H, the two carbons in this group are not included in the C₁₋₂₀alkyl carbon limitation.

When a carbon number limit is given at the beginning of a term which itself comprises two terms, the carbon number limitation is understood as inclusive for both terms. For example, for the term “C₇₋₁₄arylalkyl,” both the “aryl” and the “alkyl” portions of the term are included the carbon count, a maximum of 14 in this example, but additional substituent groups thereon are not included in the atom count unless they incorporate a carbon from the group's designated carbon count, as in the “oxo” example above. Likewise when an atom number limit is given, for example “6-14 membered heteroarylalkyl,” both the “heteroaryl” and the “alkyl” portion are included the atom count limitation, but additional substituent groups thereon are not included in the atom count unless they incorporate a carbon from the group's designated carbon count. In another example, “C₄₋₁₀cycloalkylalkyl” means a cycloalkyl bonded to the parent structure via an alkylene, alkylidene or alkylidyne; in this example the group is limited to 10 carbons inclusive of the alkylene, alkylidene or alkylidyne subunit. As another example, the “alkyl” portion of, e.g. “C₇₋₁₄arylalkyl” is meant to include alkylene, alkylidene or alkylidyne, unless stated otherwise, e.g. as in the terms “C₇₋₁₄arylalkylene” or “C₆₋₁₀aryl-CH₂CH₂—.”

“Alkylene” refers to straight, branched and cyclic (and combinations thereof) divalent radical consisting solely of carbon and hydrogen atoms, containing no unsaturation and having from one to ten carbon atoms, for example, methylene, ethylene, propylene, n-butylene and the like. Alkylene is like alkyl, referring to the same residues as alkyl, but having two points of attachment and, specifically, fully saturated. Examples of alkylene include ethylene (—CH₂CH₂—), propylene (—CH₂CH₂CH₂—), dimethylpropylene (—CH₂C(CH₃)₂CH₂—), cyclohexan-1,4-diyl and the like.

“Alkylidene” refers to straight, branched and cyclic (and combinations thereof) unsaturated divalent radical consisting solely of carbon and hydrogen atoms, having from two to ten carbon atoms, for example, ethylidene, propylidene, n-butylidene, and the like. Alkylidene is like alkyl, referring to the same residues as alkyl, but having two points of attachment and, specifically, at least one unit of double bond unsaturation. Examples of alkylidene include vinylidene (—CH═CH—), cyclohexylvinylidene (—CH═C(C₆H₁₃)—), cyclohexen-1,4-diyl and the like.

“Alkylidyne” refers to straight, branched and cyclic (and combinations thereof) unsaturated divalent radical consisting solely of carbon and hydrogen atoms having from two to ten carbon atoms, for example, propylid-2-ynyl, n-butylid-1-ynyl, and the like. Alkylidyne is like alkyl, referring to the same residues as alkyl, but having two points of attachment and, specifically, at least one unit of triple bond unsaturation.

Any of the above radicals” “alkylene,” “alkylidene” and “alkylidyne,” when optionally substituted, can contain alkyl substitution which itself can contain unsaturation. For example, 2-(2-phenylethynyl-but-3-enyl)-naphthalene (IUPAC name) contains an n-butylid-3-ynyl radical with a vinyl substituent at the 2-position of the radical. Combinations of alkyls and carbon-containing substitutions thereon are limited to thirty carbon atoms.

“Alkoxy” refers to the group —O-alkyl, where alkyl is as defined herein. Alkoxy includes, by way of example, methoxy, ethoxy, n-propoxy, isopropoxy, n-butoxy, t-butoxy, sec-butoxy, n-pentoxy, cyclohexyloxy, cyclohexenyloxy, cyclopropylmethyloxy, and the like.

“Haloalkyloxy” refers to the group —O-alkyl, where alkyl is as defined herein, and further, alkyl is substituted with one or more halogens. By way of example, a haloC₁₋₃alkyloxy” group includes —OCF₃, —OCF₂H, —OCHF₂, —OCH₂CH₂Br, —OCH₂CH₂CH₂I, —OC(CH₃)₂Br, —OCH₂Cl and the like.

“Acyl” refers to the groups —C(O)H, —C(O)alkyl, —C(O)aryl and C(O)heterocyclyl.

“α-Amino Acids” refer to naturally occurring and commercially available α-amino acids and optical isomers thereof. Typical natural and commercially available α-amino acids are glycine, alanine, serine, homoserine, threonine, valine, norvaline, leucine, isoleucine, norleucine, aspartic acid, glutamic acid, lysine, omithine, histidine, arginine, cysteine, homocysteine, methionine, phenylalanine, homophenylalanine, phenylglycine, ortho-tyrosine, meta-tyrosine, para-tyrosine, tryptophan, glutamine, asparagine, proline and hydroxyproline. A “side chain of an α-amino acid” refers to the radical found on the α-carbon of an α-amino acid as defined above, for example, hydrogen (for glycine), methyl (for alanine), benzyl (for phenylalanine), etc.

“Amino” refers to the group NH₂.

“Amide” refers to the group C(O)NH₂ or —N(H)acyl.

“Aryl” (sometimes referred to as “Ar”) refers to a monovalent aromatic carbocyclic group of, unless specified otherwise, from 6 to 15 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, 9,10-dihydrophenanthrenyl, indanyl, tetralinyl, and fluorenyl and the like), provided that the point of attachment is through an atom of an aromatic portion of the aryl group and the aromatic portion at the point of attachment contains only carbons in the aromatic ring. If any aromatic ring portion contains a heteroatom, the group is a heteroaryl and not an aryl. Aryl groups are monocyclic, bicyclic, tricyclic or tetracyclic.

“Arylene” refers to an aryl that has at least two groups attached thereto. For a more specific example, “phenylene” refers to a divalent phenyl ring radical. A phenylene, thus can have more than two groups attached, but is defined by a minimum of two non-hydrogen groups attached thereto.

“Arylalkyl” refers to a residue in which an aryl moiety is attached to a parent structure via one of an alkylene, alkylidene, or alkylidyne radical. Examples include benzyl, phenethyl, phenylvinyl, phenylallyl and the like. When specified as “optionally substituted,” both the aryl, and the corresponding alkylene, alkylidene, or alkylidyne portion of an arylalkyl group can be optionally substituted. By way of example, “C₇₋₁₁arylalkyl” refers to an arylalkyl limited to a total of eleven carbons, e.g., a phenylethyl, a phenylvinyl, a phenylpentyl and a naphthylmethyl are all examples of a “C₇₋₁₁arylalkyl” group.

“Aryloxy” refers to the group —O-aryl, where aryl is as defined herein, including, by way of example, phenoxy, naphthoxy, and the like.

“Carboxyl,” “carboxy” or “carboxylate” refers to CO₂H or salts thereof.

“Carboxyl ester” or “carboxy ester” or “ester” refers to the group —CO₂alkyl, —CO₂aryl or —CO₂heterocyclyl.

“Carbonate” refers to the group —OCO₂alkyl, —OCO₂aryl or —OCO₂heterocyclyl.

“Carbamate” refers to the group —OC(O)NH₂, —N(H)carboxyl or —N(H)carboxyl ester.

“Cyano” or “nitrile” refers to the group —CN.

“Formyl” refers to the specific acyl group —C(O)H.

“Halo” or “halogen” refers to fluoro, chloro, bromo and iodo.

“Haloalkyl” and “haloaryl” refer generically to alkyl and aryl radicals that are substituted with one or more halogens, respectively. By way of example “dihaloaryl,” “dihaloalkyl,” “trihaloaryl” etc. refer to aryl and alkyl substituted with a plurality of halogens, but not necessarily a plurality of the same halogen; thus 4-chloro-3-fluorophenyl is a dihaloaryl group.

“Heteroalkyl” refers to an alkyl where one or more, but not all, carbons are replaced with a heteroatom. A heteroalkyl group has either linear or branched geometry. By way of example, a “2-6 membered heteroalkyl” is a group that can contain no more than 5 carbon atoms, because at least one of the maximum 6 atoms must be a heteroatom, and the group is linear or branched. Also, for the purposes of this invention, a heteroalkyl group always starts with a carbon atom, that is, although a heteroalkyl may contain one or more heteroatoms, the point of attachment to the parent molecule is not a heteroatom. A 2-6 membered heteroalkyl group includes, for example, —CH₂XCH₃, —CH₂CH₂XCH₃, —CH₂CH₂XCH₂CH₃, C(CH₂)₂XCH₂CH₃ and the like, where X is O, NH, NC₁₋₆alkyl and S(O)₀₋₂, for example.

“Perhalo” as a modifier means that the group so modified has all its available hydrogens replaced with halogens. An example would be “perhaloalkyl.” Perhaloalkyls include —CF₃, —CF₂CF₃, perchloroethyl and the like.

“Hydroxy” or “hydroxyl” refers to the group —OH.

“Heteroatom” refers to O, S, N, or P.

“Heterocyclyl” in the broadest sense includes aromatic and non-aromatic ring systems and more specifically refers to a stable three- to fifteen-membered ring radical that consists of carbon atoms and from one to five heteroatoms. For purposes of this description, the heterocyclyl radical can be a monocyclic, bicyclic or tricyclic ring system, which can include fused or bridged ring systems as well as spirocyclic systems; and the nitrogen, phosphorus, carbon or sulfur atoms in the heterocyclyl radical can be optionally oxidized to various oxidation states. In a specific example, the group —S(O)₀₋₂—, refers to —S— (sulfide), —S(O)— (sulfoxide), and —SO₂— (sulfone) linkages. For convenience, nitrogens, particularly but not exclusively, those defined as annular aromatic nitrogens, are meant to include their corresponding N-oxide form, although not explicitly defined as such in a particular example. Thus, for a compound having, for example, a pyridyl ring; the corresponding pyridyl-N-oxide is meant to be included in the presently disclosed compounds. In addition, annular nitrogen atoms can be optionally quaternized. “Heterocycle” includes heteroaryl and heteroalicyclyl, that is a heterocyclic ring can be partially or fully saturated or aromatic. Thus a term such as “heterocyclylalkyl” includes heteroalicyclylalkyls and heteroarylalkyls. Examples of heterocyclyl radicals include, but are not limited to, azetidinyl, acridinyl, benzodioxolyl, benzodioxanyl, benzofuranyl, carbazoyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzoxazolyl, furyl, diazabicycloheptane, diazapane, diazepine, tetrahydrofuryl, tetrahydropyranyl, thienyl, benzothieliyl, thiamorpholinyl, thiamorpholinyl sulfoxide, thiamorpholinyl sulfone, dioxaphospholanyl, and oxadiazolyl.

“Heteroaryl” refers to an aromatic group having from 1 to 10 annular carbon atoms and 1 to 4 annular heteroatoms. Heteroaryl groups have at least one aromatic ring component, but heteroaryls can be fully unsaturated or partially unsaturated. If any aromatic ring in the group has a heteroatom, then the group is a heteroaryl, even, for example, if other aromatic rings in the group have no heteroatoms. For example, 2H-pyrido[3,2-b][1,4]oxazin-3(4H)-one-7-yl, indolyl and benzimidazolyl are “heteroaryls.” Heteroaryl groups can have a single ring (e.g., pyridinyl, imidazolyl or furyl) or multiple condensed rings (e.g., indolizinyl, quinolinyl, benzimidazolyl or benzothienyl), where the condensed rings may or may not be aromatic and/or contain a heteroatom, provided that the point of attachment to the parent molecule is through an atom of the aromatic portion of the heteroaryl group. In one embodiment, the nitrogen and/or sulfur ring atom(s) of the heteroaryl group are optionally oxidized to provide for the N-oxide (N→O), sulfinyl, or sulfonyl moieties. Compounds described herein containing phosphorous, in a heterocyclic ring or not, include the oxidized forms of phosphorous. Heteroaryl groups are monocyclic, bicyclic, tricyclic or tetracyclic.

“Heteroaryloxy” refers to O-heteroaryl.

“Heteroarylene” generically refers to any heteroaryl that has at least two groups attached thereto. For a more specific example, “pyridylene” refers to a divalent pyridyl ring radical. A pyridylene, thus can have more than two groups attached, but is defined by a minimum of two non-hydrogen groups attached thereto.

“Heteroalicyclic” refers specifically to a non-aromatic heterocyclyl radical. A heteroalicyclic may contain unsaturation, but is not aromatic. As mentioned, aryls and heteroaryls are attached to the parent structure via an aromatic ring. So, e.g., 2H-1,4-benzoxazin-3(4H)-one-4-yl is a heteroalicyclic, while 2H-1,4-benzoxazin-3(4H)-one-7-yl is an aryl. In another example, 2H-pyrido[3,2-b][1,4]oxazin-3(4H)-one-4-yl is a heteroalicyclic, while 2H-pyrido[3,2-b][1,4]oxazin-3 (4H)-one-7-yl is a heteroaryl.

“Heterocyclylalkyl” refers to a heterocyclyl group linked to the parent structure via e.g an alkylene linker, for example (tetrahydrofuran-3-yl)methyl- or (pyridin-4-yl)methyl

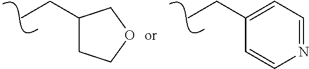

“Heterocyclyloxy” refers to the group —O-heterocycyl.

“Nitro” refers to the group —NO₂.

“Oxo” refers to a double bond oxygen radical, ═O.

“Oxy” refers to —O— radical (also designated as [symbol] O), that is, a single bond oxygen radical. By way of example, N-oxides are nitrogens bearing an oxy radical.

When a group with its bonding structure is denoted as being bonded to two partners; that is, a divalent radical, for example, —OCH₂—, then it is understood that either of the two partners can be bound to the particular group at one end, and the other partner is necessarily bound to the other end of the divalent group, unless stated explicitly otherwise. Stated another way, divalent radicals are not to be construed as limited to the depicted orientation, for example “—OCH2-” is meant to mean not only “—OCH₂—” as drawn, but also “—CH₂O—.”

When a group with its bonding structure is denoted as being bonded to two partners; that is, a divalent radical, for example, —OCH₂—, then it is understood that either of the two partners can be bound to the particular group at one end, and the other partner is necessarily bound to the other end of the divalent group, unless stated explicitly otherwise. Stated another way, divalent radicals are not to be construed as limited to the depicted orientation, for example “—OCH₂—” is meant to mean not only “—OCH₂—” as drawn, but also “—CH₂O—.”

“Optional” or “optionally” means that the subsequently described event or circumstance may or may not occur, and that the description includes instances where said event or circumstance occurs and instances in which it does not. One of ordinary skill in the art would understand that, with respect to any molecule described as containing one or more optional substituents, that only synthetically feasible compounds are meant to be included. “Optionally substituted” refers to all subsequent modifiers in a term, for example in the term “optionally substituted arylC₁₋₈alkyl,” optional substitution may occur on both the “C₁₋₈alkyl” portion and the “aryl” portion of the arylC₁₋₈alkyl group. Also by way of example, optionally substituted alkyl includes optionally substituted cycloalkyl groups. The term “substituted,” when used to modify a specified group or radical, means that one or more hydrogen atoms of the specified group or radical are each, independently of one another, replaced with the same or different substituent groups as defined below. Thus, when a group is defined as “optionally substituted” the definition is meant to encompass when the groups is substituted with one or more of the radicals defined below, and when it is not so substituted.

Substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —N(R⁸⁰)₂, perhaloalkyl, —CN, —OCN, —SCN, —NO, —NO₂, ═N₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —P(O)(O⁻)₂(M⁺)₂, —P(O)(O⁻)₂M²⁺, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)N(R⁸⁰)₂, —C(NR⁷⁰)(R⁸⁰)₂, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)N(R⁸⁰)₂, —NR⁷⁰C(NR⁷⁰)R⁷⁰ and —NR⁷⁰C(NR⁷⁰)N(R⁸⁰)₂, where R⁶⁰ is C₁₋₆alkyl, 3 to 10-membered heterocyclyl, 3 to 10-membered heterocyclylC₁₋₆alkyl, C₆₋₁₀aryl or C₆₋₁₀arylC₁₋₆alkyl; each R⁷⁰ is independently for each occurence hydrogen or R⁶⁰; each R⁸⁰ is independently for each occurence R⁷⁰ or alternatively, two R⁸⁰'s, taken together with the nitrogen atom to which they are bonded, form a 3 to 7-membered heteroalicyclyl which optionally includes from 1 to 4 of the same or different additional heteroatoms selected from O, N and S, of which N optionally has H or C₁-C₃alkyl substitution; and each M⁺ is a counter ion with a net single positive charge. Each M⁺ is independently for each occurence, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (a “subscript 0.5 means e.g. that one of the counter ions for such divalent alkali earth ions can be an ionized form of a compound described herein and the other a typical counter ion such as chloride, or two ionized compounds can serve as counter ions for such divalent alkali earth ions, or a doubly ionized compound can serve as the counter ion for such divalent alkali earth ions). As specific examples, —N(R⁸⁰)₂ is meant to include —NH₂, —NH-alkyl, —NH-pyrrolidin-3-yl, N-pyrrolidinyl, N-piperazinyl, 4N-methyl-piperazin-1-yl, N-morpholinyl and the like.

Substituent groups for replacing hydrogens on unsaturated carbon atoms in groups containing unsaturated carbons are, unless otherwise specified, —R⁶⁰, halo, —O⁻M⁺, —OR⁷⁰, —SR⁷⁰, —S⁻M⁺, —N(R⁸⁰)₂, perhaloalkyl, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —PO₃⁻²M²⁺, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)N(R⁸⁰)₂, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)N(R⁸⁰)₂, —NR⁷⁰C(NR⁷⁰)R⁷⁰ and —NR⁷⁰C(NR⁷⁰)N(R⁸⁰)₂, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

Substituent groups for replacing hydrogens on nitrogen atoms in groups containing such nitrogen atoms are, unless otherwise specified, —R⁶⁰, —O⁻M⁺, —OR⁷⁰, —SR⁷⁰, —S⁻M⁺, —N(R⁸⁰)₂, perhaloalkyl, —CN, —NO, —NO₂, —S(O)₂R⁷⁰, —SO₃⁻M⁺, —SO₃R⁷⁰, —OS(O)₂R⁷⁰, —OSO₃⁻M⁺, —OSO₃R⁷⁰, —PO₃²⁻(M⁺)₂, —PO₃²⁻M²⁺, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —CO₂R⁷⁰, —C(S)OR⁷⁰, —C(O)NR⁸⁰R⁸⁰, —C(NR⁷⁰)NR⁸⁰R⁸⁰, OC(O)R⁷⁰, —OC(S)R⁷⁰, —OCO₂R⁷⁰, —OC(S)OR⁷⁰, —NR⁷⁰C(O)R⁷⁰, —NR⁷⁰C(S)R⁷⁰, —NR⁷⁰CO₂R⁷⁰, —NR⁷⁰C(S)OR⁷⁰, —NR⁷⁰C(O)N(R⁸⁰)₂, —NR⁷⁰C(NR⁷⁰)R⁷⁰ and —NR⁷⁰C(NR⁷⁰)N(R⁸⁰)₂, where R⁶⁰, R⁷⁰, R⁸⁰ and M⁺ are as previously defined.

In one embodiment, a group that is substituted has 1, 2, 3, or 4 substituents, 1, 2, or 3 substituents, 1 or 2 substituents, or 1 substituent.

It is understood that in all substituted groups, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such case that the language permits such multiple substitutions, the maximum number of such iterations of substitution is three.

“Sulfonamide” refers to the group —SO₂NH₂, —N(H)SO₂H, —N(H)SO₂alkyl, —N(H)SO₂aryl, or —N(H)SO₂heterocyclyl.

“Sulfonyl” refers to the group —SO₂H, —SO₂alkyl, —SO₂aryl, or —SO₂heterocyclyl.

“Sulfanyl” refers to the group: —SH, —S-alkyl, —S-aryl, or —S-heterocyclyl.

“Sulfinyl” refers to the group: —S(O)H, —S(O)alkyl, —S(O)aryl or —S(O)heterocyclyl.

“Suitable leaving group” is defined as the term would be understood by one of ordinary skill in the art; that is, a group on a carbon, where upon reaction a new bond is to be formed, the carbon loses the group upon formation of the new bond. A typical example employing a suitable leaving group is a nucleophilic substitution reaction, e.g., on a sp³ hybridized carbon (SN₂ or SN₁), e.g. where the leaving group is a halide, such as a bromide, the reactant might be benzyl bromide. Another typical example of such a reaction is a nucleophilic aromatic substitution reaction (SNAr). Another example is an insertion reaction (for example by a transition metal) into the bond between an aromatic reaction partner bearing a leaving group followed by reductive coupling. “Suitable leaving group” is not limited to such mechanistic restrictions. Examples of suitable leaving groups include halogens, optionally substituted aryl or alkyl sulfonates, phosphonates, azides and —S(O)₀₋₂R where R is, for example optionally substituted alkyl, optionally substituted aryl, or optionally substituted heteroaryl. Those of skill in the art of organic synthesis will readily identify suitable leaving groups to perform a desired reaction under different reaction.

“Stereoisomer” and “stereoisomers” refer to compounds that have the same atomic connectivity but different atomic arrangement in space. Stereoisomers include cis-trans isomers, E and Z isomers, enantiomers and diastereomers. Compounds described herein, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers can be prepared using chiral synthons, chiral reagents, or resolved using conventional techniques, such as by: formation of diastereoisomeric salts or complexes which can be separated, for example, by crystallization; via formation of diastereoisomeric derivatives which can be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer can be synthesized by asymmetric synthesis using optically active reagents, substrates, catalysts or solvents, or by converting on enantiomer to the other by asymmetric transformation. For a mixture of enantiomers, enriched in a particular enantiomer, the major component enantiomer can be further enriched (with concomitant loss in yield) by recrystallization.

When the compounds described herein contain olefinic double bonds or other centers of geometric asymmetry, and unless specified otherwise, it is intended that the compounds include both E and Z geometric isomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH-ring atom arrangement, such as pyrazoles, imidazoles, benzimidazoles, triazoles, and tetrazoles. A person of ordinary skill in the art would recognize that other tautomeric ring atom arrangements are possible and contemplated herein.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. Pharmaceutically acceptable acid addition salts are those salts that retain the biological effectiveness of the free bases while formed by acid partners that are not biologically or otherwise undesirable, e.g., inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Pharmaceutically acceptable base addition salts include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.).

“Prodrug” refers to compounds that are transformed in vivo to yield the parent compound, for example, by hydrolysis in the gut or enzymatic conversion in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) where the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention can be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Laurence Brunton and Bruce Chabner, “Goodman and Gilman's The Pharmacological Basis of Therapeutics” 12^{th} Ed., 2011, McGraw-Hill, which is herein incorporated by reference). The metabolite of a compound described herein or its salt can itself be a biologically active compound in the body. While a prodrug described herein would meet this criteria, that is, form a described biologically active parent compound in vivo, “metabolite” is meant to encompass those compounds not contemplated to have lost a progroup, but rather all other compounds that are formed in vivo upon administration of a compound described herein which retain the biological activities described herein. Thus one aspect of the invention is a metabolite of a compound described herein. For example, a biologically active metabolite is discovered serendipitously, that is, no prodrug design per se was undertaken. Stated another way, biologically active compounds inherently formed as a result of practicing methods of the invention, are contemplated and disclosed herein. “Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. The compounds described herein can exist in unsolvated as well as solvated forms with solvents, pharmaceutically acceptable or not, such as water, ethanol, and the like. Solvated forms of the presently disclosed compounds are contemplated herein and are encompassed by the invention, at least in generic terms.

It is understood that the above definitions are not intended to include impermissible substitution patterns (e.g., methyl substituted with 5 fluoro groups). Such impermissible substitution patterns are easily recognized by a person having ordinary skill in the art.

### BRIEF DESCRIPTION OF THE DRAWINGS

FIGS. 1A-C illustrate that DMF increases mitochondrial copy number, mitochondrial biogenesis marker expression and mitochondrial complex expression in human fibroblasts. Human fibroblast cells were treated with 0.1% DMSO vehicle, 3 μM, 10 μM or 30 μM DMF for 48 hours. A) qPCR analysis of mitochondrial DNA copy number over nuclear DNA copy number (MT-TL1/B2M). B) qPCR analysis of TFAM normalized to β-Actin. C) qPCR analysis of two subunits of complex 1-5. Bars represent averages±standard deviations (n=3, p<0.05*, p<0.01**, p<0.001***).

FIGS. 2A-C illustrate that DMF increases basal and maximal mitochondrial oxygen consumption rates. A) Oxygen consumption rates (OCR) of human fibroblasts treated with 0.1% DMSO vehicle, 3 μM, 10 μM or 30 μM DMF for 48 hours. B) Relative basal oxygen consumption normalized to vehicle-treated fibroblasts, noted as segment B in FIG. 2A. C) Relative maximal oxygen consumption of mitochondrial uncoupled (FCCP) cells normalized to vehicle-treated fibroblasts, noted as segment C in FIG. 2A. Bars represent averages±standard deviations (n=8, p<0.05*, p<0.01**, p<0.001***).

FIGS. 3A-B illustrate that DMF increases mitochondrial copy number and mitochondrial complex expression in mouse skeletal muscle, cerebellum and liver. C57BL/6 mice were intraperitoneally injected daily with 10 mg/kg of DMF for two weeks. A) qPCR analysis of mitochondrial DNA copy number over nuclear DNA copy number (mt-Nd1/Cftr). B) qPCR analysis of the mitochondrial complex subunits mt-Nd2, mt-Co1 and mt-Atp6 normalized to β-Actin. Bars represent averages±standard deviations (n=6, p<0.05*, p<0.01**, p<0.001***).

FIGS. 4A-D illustrate that DMF increases mitochondrial copy number and mitochondrial complex expression in MS patients. PBMCs were collected from whole blood of MS patients before and after 3 months of DMF treatment and healthy individuals. A) qPCR analysis of mitochondrial DNA copy number over nuclear DNA copy number (MT-TL1/B2M) in MS patients treated with DMF relative to its own baseline. B) qPCR analysis of mitochondrial DNA copy number over nuclear DNA copy number (MT-TL1/B2M) in MS patients before and after treatment relative to healthy control group C) qPCR analysis of mitochondrial complex subunits mt-ND6, mt-CYB, mt-CO2 and mt-ATP6 in MS patients treated with DMF relative to its own baseline. D) qPCR analysis of average mitochondrial complex mRNA expression of mt-ND6, mt-CYB, mt-CO2 and mt-ATP6 in MS patients before and after treatment relative to healthy control group. Bars represent averages±standard deviations (n=11, p<0.05*, p<0.01**, p<0.001***).

FIGS. 5A-C illustrate that stimulation of mitochondrial proliferation and mitochondrial complex transcription, by DMF, is mediated by Nrf2. Human fibroblast cells were treated with control or Nrf2 siRNA for 48 hours followed by 0.1% DMSO vehicle, 3 μM, 10 μM or 30 μM DMF treatment for 48 hours. A) qPCR analysis of Nrf2, NQO1 and NRF1 normalized to β-Actin. B) qPCR analysis of mitochondrial DNA copy number over nuclear DNA copy number (MT-TL/B2M). C) qPCR analysis of MT-ND2 (complex 1), SDHB (complex 2), CYC1 (complex 3), MT-CO2 (complex 4), and ATP5B (complex 5) normalized to β-Actin. Bars represent averages±standard deviations (n=3, p<0.05*, p<0.01**, p<0.001***).

FIGS. 6A-C illustrate that stimulation of mitochondrial proliferation and mitochondrial complex 2-5 transcription, by DMF, is not mediated by HCAR2. Human fibroblast cells were treated with control or HCAR2 siRNA for 48 hours followed by 0.1% DMSO vehicle, 3 μM, 10 μM or 30 μM DMF treatment for 48 hours. A) qPCR analysis of NFE2L2, NQO1 and NRF1 normalized to β-Actin. B) qPCR analysis of mitochondrial DNA copy number over nuclear DNA copy number (MT-TL/B2M). C) qPCR analysis of MT-ND2 (complex 1), SDHB (complex 2), CYC1 (complex 3), MT-CO2 (complex 4), and ATP5B (complex 5) normalized to 13-Actin. Bars represent averages±standard deviations (n=3, p<0.05*, p<0.01**, p<0.001***).

FIG. 7 illustrates a mechanistic diagram of dimethyl fumarate-induced mitochondrial biogenesis.

### DETAILED DESCRIPTION

### 1. Introduction

Mitochondrial mass and functionality decreases in multiple contexts, including muscle wasting diseases, muscular dystrophies, cancer cachexia, and age-related muscle wasting (sarcopenia). The consequences of decreased mitochondrial mass in muscle include increased susceptibility to falls, necessity for a wheelchair, and increased frailty. Mitochondrial mass and functionality also declines in the context of liver disorders. We observe that dimethylfumarate (DMF) dose-dependently increases mitochondrial biogenesis, mass and functionality (e.g., mitochondrial oxidative respiration and gene expression) in muscle and liver tissues, and is therefore of benefit to those affected with muscle and liver disorders.

Dimethyl fumarate (DMF) is a methyl ester of fumaric acid with known anti-inflammatory properties. DMF is currently being used to treat multiple sclerosis and psoriasis under the name Tecfidera and Fumaderm, respectively. We have identified a new function and use for DMF, alone and in combination with methylene blue, for increasing mitochondrial mass, numbers and/or functionality, e.g, for the amelioration of muscle and liver disorders. The only known targets of DMF and methylene blue are Nrf2/Keap1 and HCA2. Without being bound to theory, the likely mechanism of action for DMF in this context is DMF→Nrf2 or HCA2→TFAM→mitochondrial biogenesis.

The induction of mitochondrial biogenesis can alleviate mitochondrial and muscle disease. We show herein that dimethyl fumarate (DMF) dose-dependently induces mitochondrial biogenesis and function dosed to cells in in vitro, and also dosed in vivo to mice and humans. The induction of mitochondrial gene expression is more dependent on its target Nrf2 than hydroxycarboxylic acid receptor 2 (HCAR2). Thus, DMF induces mitochondrial biogenesis primarily through its action on Nrf2, and is the first drug demonstrated to increase mitochondrial biogenesis with in vivo human dosing. The observation that DMF stimulates mitochondrial biogenesis, gene expression and function demonstrates its use for mitochondrial disease therapy and/or therapy in muscle disease in which mitochondrial function is important.

As a consequence of screening drugs for effect on mitochondrial functions a group of mitoactive drugs were identified including DMF (28). We studied the effects of DMF on mitochondria in human fibroblasts, C57BL/6 mice and human MS patients. We report a novel mitochondrial biogenesis effect of DMF; to increase mitochondrial copy number and expression of mitochondria complexes in vitro and in vivo, in cells and mice and humans. Furthermore, we note a mitochondrial gene expression deficit in human MS patients, that can be used as a biomarker of disease severity. Mechanistically by knockdown, we show that DMF's mitochondrial biogenesis effect is attributable to Nrf2 rather than HCAR2.

Currently, there is no FDA-approved drug for mitochondrial disease, and a drug that increases mitochondrial biogenesis can ameliorate symptoms of muscle diseases as well. DMF is already approved for other indications, and because it increases mitochondrial mass and activity in vitro and in vivo, it can ameliorate symptoms for diseases associated with or caused by mitochondrial deficiency.

### 2. Subjects Who May Benefit

Subjects who may benefit from methods that increase the mitochondrial mass, number and function generally have a muscle disorder (e.g., a muscle wasting syndrome) and/or a liver dysfunction disorder. Illustrative muscle disorders and liver dysfunction disorders include without limitation those listed below in the next section and herein. The subject may be actively manifesting symptoms, or the symptoms may be suppressed or controlled (e.g., by medication) or in remission. The subject may or may not have been diagnosed with the disorder, e.g., by a qualified medical practitioner. In varying embodiments, the subject is already receiving a treatment regime for the muscle disorder or the liver dysfunction disorder).

In varying embodiments, the subject is a child, a juvenile or an adult. In varying embodiments, the subject is a mammal, for example, a human, a non-human primate or a domesticated mammal (e.g., a canine or a feline).

### 3. Conditions Subject to Treatment

a. Muscle Disorders

In varying embodiments the subject has a muscle disorder that is associated with or caused at least in part due to deficient mitochondrial mass, numbers and/or function. In varying embodiments, the muscle disorder affects the mass, strength and/or function of skeletal muscle. In some embodiments, the muscle disorder is a muscle wasting syndrome. Illustrative muscle disorders for which one or more symptoms can be mitigated, ameliorated, reduced, inhibited and/or eliminated by the present methods include without limitation Cancer cachexia, age-related muscle wasting (sarcopenia), Mitochondrial myopathy, Acid Maltase Deficiency (AMD), Amyotrophic Lateral Sclerosis (ALS), Amyotrophy, Andersen-Tawil Syndrome, Anterior compartment syndrome of the lower leg, Becker Muscular Dystrophy (BMD), Becker Myotonia Congenita, Bethlem Myopathy, Bimagrumab, Bulbospinal Muscular Atrophy (Spinal-Bulbar Muscular Atrophy), Carnitine Deficiency, Carnitine Palmityl Transferase Deficiency (CPT Deficiency), Cataplexy, Central core disease of muscle, Centronuclear Myopathy, Charcot-Marie-Tooth Disease (CMT), Charley horse, Chronic fatigue syndrome, Chronic progressive external ophthalmoplegia, Congenital Muscular Dystrophy (CMD), Congenital Myasthenic Syndromes (CMS), Congenital Myotonic Dystrophy, Contracture, Cori Disease (Debrancher Enzyme Deficiency), Cramp, Cricopharyngeal spasm, Debrancher Enzyme Deficiency, Dejerine-Sottas Disease (DSD), Dermatomyositis (DM), Diastasis recti, Distal Muscular Dystrophy (DD), Distal spinal muscular atrophy type 2, Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Exercise therapy for idiopathic inflammatory myopathies, Exercise-associated muscle cramps, Exertional rhabdomyolysis, Facioscapulohumeral Muscular Dystrophy (FSH or FSHD), Fibrodysplasia ossificans progressive, Finnish (Tibial) Distal Myopathy, Forbes Disease (Debrancher Enzyme Deficiency), Fukuyama Congenital Muscular Dystrophy, Glycogen storage disease type XI, Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery-Dreifuss Muscular Dystrophy), Hereditary inclusion body myopathy and myositis, Hereditary Motor and Sensory Neuropathy (Charcot-Marie-Tooth Disease), Hyperthyroid Myopathy, Hypertonia, Hypothyroid Myopathy, Inclusion-Body Myositis (IBM) and myopathy, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Laminopathy, Late-onset mitochondrial myopathy, Limb-Girdle Muscular Dystrophy (LGMD), Lou Gehrig's Disease (Amyotrophic Lateral Sclerosis), Macrophagic myofasciitis, McArdle Disease (Phosphorylase Deficiency), Merosin-Deficient Congenital Muscular Dystrophy, Metabolic myopathy, Mitochondrial Myopathy, Miyoshi Distal Myopathy, Motor Neurone Disease, Muscle atrophy, Muscle fatigue, Muscle imbalance, Muscle weakness, Muscle-Eye-Brain Disease, Myasthenia Gravis (MG), Myoadenylate Deaminase Deficiency, Myofibrillar Myopathy, Myopathy, Myopathy, X-linked, with excessive autophagy, Myophosphorylase Deficiency, Myositis, Myositis ossificans, Myostatin-related muscle hypertrophy, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Orofacial myological disorders, Paramyotonia Congenita, Paratonia, Pearson Syndrome, Pelvic floor muscle disorder, Periodic Paralysis, Peroneal Muscular Atrophy (Charcot-Marie-Tooth Disease), Phosphofructokinase Deficiency, Phosphoglycerate Kinase Deficiency, Phosphorylase Deficiency, Polymyositis (PM), Pompe Disease (Acid Maltase Deficiency), Progressive External Ophthalmoplegia (PEO), Psoas muscle abscess, Pyomyositis, Rod Body Disease (Nemaline Myopathy), Sarcoglycanopathy, Sphincter paralysis, Spinal Muscular Atrophy (SMA), Spinal-Bulbar Muscular Atrophy (SBMA)/Kennedy's disease, Steinert Disease (Myotonic Muscular Dystrophy), Strain (injury), Tarui Disease (Phosphofructokinase Deficiency), Thomsen Disease (Myotonia Congenita), Thyrotoxic periodic paralysis, Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), ZASP-Related Myopathy and Zenker's degeneration.

In varying embodiments, the muscle disorder is a muscular dystrophy, e.g., Becker Muscular Dystrophy (BMD), Congenital Muscular Dystrophy (CMD), Congenital Myotonic Dystrophy, Distal Muscular Dystrophy (DD), Duchenne Muscular Dystrophy (DMD), Dystrophia Myotonica (Myotonic Muscular Dystrophy), Emery-Dreifuss Muscular Dystrophy (EDMD), Facioscapulohumeral Muscular Dystrophy (FSH or FSHD), Fukuyama Congenital Muscular Dystrophy, Hauptmann-Thanheuser MD (Emery-Dreifuss Muscular Dystrophy), Merosin-Deficient Congenital Muscular Dystrophy, Integrin-Deficient Congenital Muscular Dystrophy, Limb-Girdle Muscular Dystrophy (LGMD), Myotonic Muscular Dystrophy (MMD), Oculopharyngeal Muscular Dystrophy (OPMD), Steinert Disease (Myotonic Muscular Dystrophy), Ullrich Congenital Muscular Dystrophy and Walker-Warburg Syndrome (Congenital Muscular Dystrophy).

In varying embodiments, the muscle disorder is a myopathy, e.g., Mitochondrial myopathy, Bethlem Myopathy, Centronuclear Myopathy, Finnish (Tibial) Distal Myopathy, Gowers-Laing Distal Myopathy, Hereditary inclusion body myopathy and myositis, Hyperthyroid Myopathy, Inclusion-Body Myositis (IBM) and myopathy, Late-onset mitochondrial myopathy, Metabolic myopathy, Mitochondrial Myopathy, Miyoshi Distal Myopathy, Myofibrillar Myopathy, Myopathy, X-linked Myopathy with excessive autophagy, Myotubular Myopathy (MTM or MM), Nemaline Myopathy, Nonaka Distal Myopathy, Welander Distal Myopathy, ZASP-Related Myopathy.

In varying embodiments, the muscle disorder is a muscle atrophy disorder, e.g., Amyotrophy, Bulbospinal Muscular Atrophy (Spinal-Bulbar Muscular Atrophy), Distal spinal muscular atrophy type 2, Kennedy Disease (Spinal-Bulbar Muscular Atrophy), Kugelberg-Welander Disease (Spinal Muscular Atrophy), Muscle atrophy, Peroneal Muscular Atrophy (Charcot-Marie-Tooth Disease), Spinal Muscular Atrophy (SMA), Spinal-Bulbar Muscular Atrophy (SBMA)/Kennedy's disease, Werdnig-Hoffmann Disease (Spinal Muscular Atrophy).

Muscle disorders that can be mitigated, ameliorated, even reversed or treated, by the present methods are known in the art and described in the literature, including, e.g., in Barnes, et al., Myopathies in Clinical Practice, 1st Edition, 2003, CRC Press and Amato and Russell, Neuromuscular Disorders, 2^{nd} Edition, 2015, McGraw-Hill Education/Medical. Additional muscle disorders subject to treatment by the present methods are described on the internet at Medscape.com.

b. Liver Disorders

In varying embodiments the subject has a liver disorder that is associated with or caused at least in part due to deficient mitochondrial mass, numbers and/or function. Illustrative liver disorders for which one or more symptoms can be mitigated, ameliorated, reduced, inhibited and/or eliminated by the present methods include without limitation mitochondrial liver disease, hepatitis, alcoholic liver disease, fatty liver disease (hepatic steatosis), NASH-Non-alcoholic steatohepatitis, Gilbert's syndrome, cirrhosis, primary liver cancer, primary biliary cirrhosis, primary sclerosing cholangitis, and Budd-Chiari syndrome.

Liver disorders that can be mitigated, ameliorated, even reversed or treated, by the present methods are known in the art and described in the literature, including, e.g., in Sanyal, et al., Zakim and Boyer's Hepatology: A Textbook of Liver Disease, 6th Edition, 2011, Saunders and Mount Sinai Expert Guides: Hepatology 1st Edition, 2014, Ahmad, et al., eds., Wiley-Blackwell.

### 4. Compounds for Administration

The methods entail contacting a myocyte and/or hepatocyte or administering to a subject in need thereof a therapeutically effective amount of methylene blue and/or a compound of Formula (I) or a pharmaceutically acceptable salt thereof; wherein R¹ and R² are independently selected from —CH₃, —OH, —O, -E, and C1-C8 alkoxy (branched or unbranched provided that at least one of R¹ and R² is C1-C8 alkoxy:

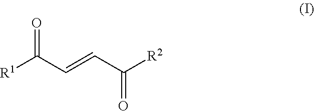

under conditions sufficient to increase mitochondrial mass and/or functionality in a mammalian myocyte and/or hepatocyte. Compounds of Formula (I) are considered to include cis and trans isomers, stereoisomers as well as optical isomers, e.g. mixtures of enantiomers as well as individual enantiomers and diastereomers, which arise as a consequence of structural asymmetry in selected compounds of the present series. Formula (I) compounds include trans (fumarate) and cis (maleate) isomers. E is an electron withdrawing group. Examples of electron withdrawing groups include —NO₂, —N(R₂), —N(R₃)\—N(H3)\—SO₃H, —SO₃R′, —S(O₂)R′ (sulfone), —S(O)R′ (sulfoxide), —S(O₂)NH₂ (sulfonamide), —SO₂NHR′, —SO₂NR′₂, —PO(OR′h, —PO₃H₂, —PO(NR′2h, pyridinyl (2-, 3-, 4-), pyrazolyl, indazolyl, imidazolyl, thiazolyl, benzothiazolyl, oxazolyl, benzimidazolyl, benzoxazolyl, isoxazolyl, benzisoxazolyl, triazolyl, benzotriazolyl, quinolinyl, isoquinolinyl, quinazolinyl, pyrimidinyl, a 5 or 6-membered heteroaryl with a C—N double bond optionally fused to a 5 or 6 membered heteroaryl, pyridinyl N-oxide, —C═N, —CX′₃, —C(O)X′, —COOH, —COOR′, —C(O)R′, —C(O)NH₂, —C(O)NHR′, —C(O)NR′₂, —C(O)H, —P(O)(OR′)OR″ and X′, wherein X′ is independently halogen (e.g. F, Cl, Br, I) and R, R′ and R″ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or similar substituents (e.g. a substituent group, a size limited substituent group or a lower substituent group).

In varying embodiments, the compound of Formula (I) comprises a fumarate ester. In varying embodiments, the compound of Formula (I) is selected from the group consisting of monomethyl fumarate (MMF), monomethyl maleate, monoethyl fumarate, monoethyl maleate, monobutyl fumarate, monobutyl maleate, monooctyl fumarate, monoctyl maleate, mono (phenylmethyl) fumarate, mono (phenylmethyl) maleate, mono (2-hydroxypropyl) fumarate, mono (2-hydroxypropyl) maleate, mono (2-ethylhexyl) fumarate, mono (2-ethylhexyl) maleate, dimethylfumarate, dimethyl maleate, diethyl fumarate, diethyl maleate, dipropyl fumarate, dipropyl maleate, diisopropyl fumarate, diisopropyl maleate, dibutyl fumarate, dibutyl maleate, diisobutyl fumarate, diisobutyl maleate, diheptyl fumarate, diheptyl maleate, bis (2-ethylhexyl) fumarate, bis (2-ethylhexyl) maleate, (−)-Dimenthyl fumarate, (−)-Bis ((S)-1-(ethoxycarbonyl)ethyl) fumarate, (−)-Bis ((S)-1-(ethoxycarbonyl)ethyl) maleate, Bis (2-trifluoroethyl) fumarate, Bis (2-trifluoroethyl) maleate, and mixtures thereof.

In varying embodiments, the compound of Formula (I) comprises dimethyl fumarate (DMF). The chemical structure of DMF is provided below:

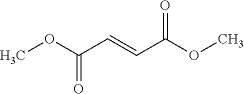

### 5. Methods of Treating Muscle and Liver Disorders

In various methods of treatment, the subject may already exhibit symptoms of disease or be diagnosed as having disease. For example, the subject may exhibit symptoms of a muscle or liver disorder, as described herein, or be diagnosed as having a muscle or liver disorder. In such cases, administration of the compound (e.g., a compound of Formula (I), e.g., dimethyl fumarate and/or methylene blue) and/or analogs thereof can reverse or delay progression of and or reduce the severity of disease symptoms.

Measurable parameters for evaluating the effectiveness of the treatment regime are as discussed herein for therapy and monitoring.

### 6. Formulation and Administration

a. Formulation

The compound (e.g., a compound of Formula (I), e.g., dimethyl fumarate and/or methylene blue) and/or an analog thereof can be administered orally, parenterally, (intravenously (IV), intramuscularly (IM), depo-IM, subcutaneously (SQ), and depo-SQ), sublingually, intranasally (inhalation), intrathecally, transdermally (e.g., via transdermal patch), topically, ionophoretically or rectally. Typically the dosage form is selected to facilitate delivery to the muscle or liver. Dosage forms known to those of skill in the art are suitable for delivery of the compound.

Compositions are provided that contain therapeutically effective amounts of the compound. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

These active agents (e.g., a compound of Formula (I), e.g., dimethyl fumarate and/or methylene blue) can be administered in the “native” form or, if desired, in the form of salts, esters, amides, prodrugs, derivatives, and the like, provided the salt, ester, amide, prodrug or derivative is suitable pharmacologically effective, e.g., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art. For example, the disulfide salts of a number of delivery agents are described in PCT Publication WO 2000/059863 which is incorporated herein by reference. Similarly, acid salts of therapeutic peptides, peptoids, or other mimetics, and can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, orotic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. In certain embodiments basic salts include alkali metal salts, e.g., the sodium salt, and copper salts.

For the preparation of salt forms of basic drugs, the pKa of the counterion is preferably at least about 2 pH lower than the pKa of the drug. Similarly, for the preparation of salt forms of acidic drugs, the pKa of the counterion is preferably at least about 2 pH higher than the pKa of the drug. This permits the counterion to bring the solution's pH to a level lower than the pHmax to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (e.g., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

In various embodiments preparation of esters typically involves functionalization of hydroxyl and/or carboxyl groups that are present within the molecular structure of the active agent. In certain embodiments, the esters are typically acyl-substituted derivatives of free alcohol groups, e.g., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

About 1 to 1000 mg of a compound or mixture of the compound (e.g., a compound of Formula (I), e.g., dimethyl fumarate and/or methylene blue) or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 1-1000 mg, 2-800 mg, 5-500 mg, 10-400 mg, 50-200 mg, e.g., about 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 200 mg, 300 mg, 400 mg, 500 mg, 600 mg, 700 mg, 800 mg, 900 mg or 1000 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, the compound is mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween™, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered and/or that is effective in a prophylactic context. Typically, the compositions are formulated for single dosage (e.g., daily) administration.

The compounds may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder. A therapeutically or prophylactically effective dose can be determined by first administering a low dose, and then incrementally increasing until a dose is reached that achieves the desired effect with minimal or no undesired side effects.

In various embodiments, the agents (e.g., a compound of Formula (I), e.g., dimethyl fumarate and/or methylene blue) are dissolved or suspended in a cyclodextrin. In varying embodiments, the cyclodextrin is an α-cyclodextrin, a β-cyclodextrin or a γ-cyclodextrin. In varying embodiments, the cyclodextrin is selected from the group consisting of hydroxypropyl-β-cyclodextrin, endotoxin controlled β-cyclodextrin sulfobutyl ethers, or cyclodextrin sodium salts (e.g., CAPTISOL®). Such formulations are useful for oral, intramuscular, intravenous and/or subcutaneous administration.

In various embodiments, the compounds and/or analogs thereof can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compounds. The containers are preferably adapted for the desired mode of administration, including, but not limited to tablets, gel capsules, sustained-release capsules, and the like for oral administration; depot products, pre-filled syringes, ampules, vials, and the like for parenteral administration; and patches, medipads, creams, and the like for topical administration.

The concentration and/or amount of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound can be provided in a formulation that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

In various embodiments, the tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

In powders, the carrier is a finely divided solid which is in a mixture with the finely divided active component. In tablets, the active component is mixed with the carrier having the necessary binding properties in suitable proportions and compacted in the shape and size desired. The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

b. Routes of Administration and Dosing

In various embodiments, the compounds and/or analogs thereof can be administered orally, parenterally (IV, IM, depo-IM, SQ, and depo-SQ), sublingually, intranasally (inhalation), intrathecally, transdermally (e.g., via transdermal patch), topically, or rectally. Dosage forms known to those skilled in the art are suitable for delivery of the compounds and/or analogs thereof.

In various embodiments, the compounds and/or analogs thereof may be administered enterally or parenterally. When administered orally, the compounds can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compound needs to be administered only once or twice daily.

The oral dosage forms can be administered to the patient 1, 2, 3, or 4 times daily. It is preferred that the compound be administered either three or fewer times, more preferably once or twice daily. Hence, it is preferred that the compound be administered in oral dosage form. It is preferred that whatever oral dosage form is used, that it be designed so as to protect the compound from the acidic environment of the stomach. Enteric coated tablets are well known to those skilled in the art. In addition, capsules filled with small spheres each coated to protect from the acidic stomach, are also well known to those skilled in the art.

When administered orally, an administered amount therapeutically effective to ameliorate, mitigate, reduce, inhibit and/or reverse one or more symptoms of a muscle or liver disorder is from about 0.1 mg/day to about 200 mg/day, for example, from about 1 mg/day to about 100 mg/day, for example, from about 5 mg/day to about 50 mg/day. In some embodiments, the subject is administered the compound at a dose of about 0.05 to about 0.50 mg/kg, for example, about 0.05 mg/kg, 0.10 mg/kg, 0.20 mg/kg, 0.33 mg/kg, 0.50 mg/kg. It is understood that while a patient may be started at one dose, that dose may be varied (increased or decreased, as appropriate) over time as the patient's condition changes. Depending on outcome evaluations, higher doses may be used. For example, in certain embodiments, up to as much as 1000 mg/day can be administered, e.g., 5 mg/day, 10 mg/day, 25 mg/day, 50 mg/day, 100 mg/day, 200 mg/day, 300 mg/day, 400 mg/day, 500 mg/day, 600 mg/day, 700 mg/day, 800 mg/day, 900 mg/day or 1000 mg/day.

The compounds and/or analogs thereof may also be advantageously delivered in a nano crystal dispersion formulation. Preparation of such formulations is described, for example, in U.S. Pat. No. 5,145,684. Nano crystalline dispersions of HIV protease inhibitors and their method of use are described in U.S. Pat. No. 6,045,829. The nano crystalline formulations typically afford greater bioavailability of drug compounds.

In various embodiments, the compounds and/or analogs thereof can be administered parenterally, for example, by IV, IM, depo-IM, SC, or depo-SC. When administered parenterally, a therapeutically effective amount of about 0.5 to about 100 mg/day, preferably from about 5 to about 50 mg daily should be delivered. When a depot formulation is used for injection once a month or once every two weeks, the dose should be about 0.5 mg/day to about 50 mg/day, or a monthly dose of from about 15 mg to about 1,500 mg. In part because of the forgetfulness of the patients with Alzheimer's disease, it is preferred that the parenteral dosage form be a depo formulation.

In various embodiments, the compounds and/or analogs thereof can be administered sublingually. When given sublingually, the compounds and/or analogs thereof can be given one to four times daily in the amounts described above for IM administration.

In various embodiments, the compounds and/or analogs thereof can be administered intranasally. When given by this route, the appropriate dosage forms are a nasal spray or dry powder, as is known to those skilled in the art. The dosage of compound and/or analog thereof for intranasal administration is the amount described above for IM administration.

In various embodiments, compound and/or analogs thereof can be administered intrathecally. When given by this route the appropriate dosage form can be a parenteral dosage form as is known to those skilled in the art. The dosage of compound and/or analog thereof for intrathecal administration is the amount described above for IM administration.

In certain embodiments, the compound and/or analog thereof can be administered topically. When given by this route, the appropriate dosage form is a cream, ointment, or patch. When administered topically, the dosage is from about 1.0 mg/day to about 200 mg/day. Because the amount that can be delivered by a patch is limited, two or more patches may be used. The number and size of the patch is not important, what is important is that a therapeutically effective amount of compound be delivered as is known to those skilled in the art. The compound can be administered rectally by suppository as is known to those skilled in the art. When administered by suppository, the therapeutically effective amount is from about 1.0 mg to about 500 mg.

In various embodiments, the compound and/or analog thereof can be administered by implants as is known to those skilled in the art. When administering the compound by implant, the therapeutically effective amount is the amount described above for depot administration.

It should be apparent to one skilled in the art that the exact dosage and frequency of administration will depend on the particular condition being treated, the severity of the condition being treated, the age, weight, general physical condition of the particular patient, and other medication the individual may be taking as is well known to administering physicians who are skilled in this art.

### 7. Methods of Monitoring

In various embodiments, the effectiveness of treatment can be determined by comparing a baseline measure of a parameter of disease before administration of the compound (e.g., a compound of Formula (I), e.g., dimethyl fumarate and/or methylene blue) and/or analogs thereof is commenced to the same parameter one or more timepoints after the compound or analog has been administered. One illustrative parameter that can be measured is a biomarker indicative of mitochondrial biogenesis, numbers and/or function (e.g., mitochondrial gene expression and/or basal mitochondrial oxidative respiration). Such biomarkers include, but are not limited to mitochondrial DNA to nuclear DNA ratio (mtDNA/nDNA ratio), increased levels of mitochondrial transcription factor A (TFAM) as an indicator of mitochondrial biogenesis and/or one or more of mitochondrial complex 1 subunits ND2 and ND6; mitochondrial complex 2 subunits SDHA and SDHB, mitochondrial complex 3 subunits mt-CYB and mt-CYC1, mitochondrial complex 4 subunits mt-CO1 and mt-CO2, and ATP synthase subunits mt-ATP5B and mt-ATP6 as an indicator of mitochondrial gene expression. Increased mitochondrial biogenesis, numbers and/or function (e.g., mitochondrial gene expression and/or basal mitochondrial oxidative respiration) is an indicator that the treatment is effective. Conversely, detection of decreased levels of mitochondrial biogenesis, numbers and/or function (e.g., mitochondrial gene expression and/or basal mitochondrial oxidative respiration) is an indicator that the treatment is not effective.

Another parameter to determine effectiveness of treatment is determination of increased muscle mass and/or strength in the case of a muscle disorder. Muscle mass, strength and function can be assessed using methods known in the art. For example, muscle strength can be being assessed by manual muscle testing, handheld myometry and quantitative muscle testing. Standard liver function tests and liver enzyme tests known in the art can be performed in the case of a liver disorder. Liver function tests established in the art include serum bilirubin test, serum albumin test and International normalized ratio (INR), also called prothrombin time (PT) test Elevated levels of bilirubin may indicate an obstruction of bile flow or a problem in the processing of bile by the liver. The PT test measures how long it takes for blood to clot Blood clotting requires vitamin K and a protein that is made by the liver. Prolonged clotting may indicate liver disease. Liver enzyme tests established in the art include Serum alkaline phosphatase test, alanine transaminase (ALT) test, aspartate transaminase (AST) test, gamma-glutamyl transpeptidase test, lactic dehydrogenase test and 5′-nucleotidase test. Alanine transaminase and aspartate transaminase are released into the bloodstream after acute liver cell damage. 5′-nucleotidase level is elevated in persons with liver diseases.

Clinical efficacy can be monitored using any method known in the art. Detection of biomarkers and clinical parameters indicative of increased mitochondrial mass, numbers and/or function and/or clinical parameters of increased muscle mass, strength and/or function and/or increased liver function are indicators that the treatment or prevention regime is efficacious. Conversely, detection of biomarkers and clinical parameters indicative of decreased mitochondrial mass, numbers and/or function and/or clinical parameters of decreased muscle mass, strength and/or function and/or decreased liver function are indicators that the treatment or prevention regime is not efficacious.

In certain embodiments, the monitoring methods can entail determining a baseline value of a measurable biomarker or parameter in a subject before administering a dosage of the compound, and comparing this with a value for the same measurable biomarker or parameter after treatment.

In other methods, a control value (e.g., a mean and standard deviation) of the measurable biomarker or clinical parameter is determined for a control population. In certain embodiments, the individuals in the control population have not received prior treatment and do not have a muscle or liver disorder, nor are at risk of developing a muscle or liver disorder. In such cases, if the value of the measurable biomarker or clinical parameter approaches the control value, then treatment is considered efficacious. In other embodiments, the individuals in the control population have not received prior treatment and have been diagnosed with a muscle disorder or a liver disorder. In such cases, if the value of the measurable biomarker or clinical parameter approaches the control value, then treatment is considered inefficacious.

In other methods, a subject who is not presently receiving treatment but has undergone a previous course of treatment is monitored for one or more of the biomarkers or clinical parameters to determine whether a resumption of treatment is required. The measured value of one or more of the biomarkers or clinical parameters in the subject can be compared with a value previously achieved in the subject after a previous course of treatment. Alternatively, the value measured in the subject can be compared with a control value (mean plus standard deviation/ANOVA) determined in population of subjects after undergoing a course of treatment. Alternatively, the measured value in the subject can be compared with a control value in populations of prophylactically treated subjects who remain free of symptoms of disease, or populations of therapeutically treated subjects who show amelioration of disease characteristics. In such cases, if the value of the measurable biomarker or clinical parameter approaches the control value, then treatment is considered efficacious and need not be resumed. In all of these cases, a significant difference relative to the control level (e.g., more than a standard deviation) is an indicator that treatment should be resumed in the subject.

The tissue sample for analysis is typically blood, plasma, serum, saliva, urine, mucous or cerebrospinal fluid from the subject. In some embodiments, the tissue sample is a biopsy of muscle tissue (e.g., skeletal muscle) or liver tissue.

### 8. Kits

Further provided are kits. In varying embodiments, the kits comprise a compound of Formula (I), e.g., dimethyl fumarate and methylene blue. Embodiments of compounds of Formula (I) are as described above and herein. Embodiments of formulations of the compounds are as described above and herein. In varying embodiments, the compound of Formula (I), e.g., dimethyl fumarate and methylene blue can be co-formulated for administration as a single composition. In some embodiments, the compound of Formula (I), e.g., dimethyl fumarate and methylene blue are formulated for separate administration, e.g., via the same or different route of administration. In varying embodiments, one or both the compound of Formula (I), e.g., dimethyl fumarate and methylene blue are provided in unitary dosages in the kits.

### EXAMPLES

The following examples are offered to illustrate, but not to limit the claimed invention.

### Example 1

### Dimethyl Fumarate Mediates Nrf2-Dependent Mitochondrial Biogenesis in Mice and Humans

### Abstract:

We show in this example that dimethyl fumarate (DMF) dose-dependently induces mitochondrial biogenesis and function dosed to cells in in vitro, and also dosed in vivo to mice and humans. The induction of mitochondrial gene expression is more dependent on its target Nrf2 than hydroxycarboxylic acid receptor 2 (HCAR2). Thus, DMF induces mitochondrial biogenesis primarily through its action on Nrf2, and is the first drug demonstrated to increase mitochondrial biogenesis with in vivo human dosing. The observation that DMF stimulates mitochondrial biogenesis, gene expression and function suggests that it could be considered for mitochondrial disease therapy and/or therapy in muscle disease in which mitochondrial function is important.

### Materials and Methods:

Fibroblast Cell Culture and Drug Treatment.

The healthy human fibroblast cell line AG09429 (Coriell Institute, Camden, N.J., USA) was maintained at 37° C. in a humidified atmosphere with 5% CO2. DMEM (Corning, Inc., Corning, N.Y., USA) supplemented with 10% fetal bovine serum (JR-Scientific, Woodland, Calif., USA), 1× Penicillin-Streptomycin Solution (Corning, Inc., Corning, N.Y., USA) was used as growth media. Media was changed every two days.

The human fibroblasts were plated in a 12-well format at 0.1×10⁶ cells per well. The cells were incubated with 0.1% DMSO as vehicle control or 3-30 μM of dimethyl fumarate (Sigma-Aldrich, St. Louis, Mo., USA) dissolved in DMSO. Total RNA and DNA were extracted following a 48-hour incubation period.

Patient Consent and Blood Collection.

The local Ethics Committee of the University Federico II of Naples, approved the study. Patients were recruited from the Multiple Sclerosis Center of the Federico II University of Naples. All patients gave written informed consent before any activity linked to the study was started. Healthy controls were recruited at the clinic through students and site personnel. The study included 12 MS patients and 11 healthy individuals. Samples were obtained on the day before and after 3 months of continuous DMF treatment. Whole blood was collected in EDTA containing Leucosep® tubes (Greiner bio-one, Frickenhausen, Germany) and frozen at −80° C. until analysis. PBMCs were isolated from 30 mL of EDTA anticoagulated whole blood for RNA extraction.

Nrf2 and HCAR2 siRNA Knockdown in Human Fibroblast Cells.

Fibroblast cells were seeded in six-well plates at 0.2×10⁶ cells per well and transfected with negative control siRNA (cat. 12935300, Thermo-Fisher, Waltham, Mass., USA), pooled Nrf2 siRNA (cat. HSS107130, HSS181505, HSS181506, Thermo-Fisher, Waltham, Mass., USA) or pooled HCAR2 siRNA (cat. L-006688-02-0005, Dharmacon, Lafayette, Colo., USA) using Lipofectamine RNAiMAX following manufacturer's instruction. After a 48-hour incubation, subsequent drug treatment was conducted with the cells.

Mouse Models, Drug Treatment, and Dissection.

C57BL/6 wild-type mice were housed in a vivarium maintained at 22° C.-24° C. and 40%-60% relative humidity with a 12-hour light/12-hour dark cycle. All experimental procedures were approved by the University of California Institutional Animal Care and Use Committee.

The stock dimethyl fumarate solution was made by dissolving 50 mg/ml of DMF into DMSO. Prior to injection, 0.5 mg/ml of working DMF solution (1:100 dilution) was made by diluting the stock solution into phosphate-buffered saline with 5% Tween-20 and 5% Polyethylene glycol (Sigma-Aldrich, St. Louis, Mo., USA). The mice were injected intraperitoneally every day for 14 days with 10 mg/kg of DMF.

The mice were euthanized with CO2 followed by cervical dislocation and tissues were immediately removed then flash frozen with liquid nitrogen. Samples were stored in −80° C. until utilized for experiments.

DNA and RNA Extraction.

Total DNA was extracted from human fibroblast and mouse tissues using DNeasy plus mini kit and DNeasy blood & tissue kit (Qiagen, Valencia, Calif., USA), respectively, following manufacturer's instruction. DNA was quantified by a NanoDrop 2000c Spectrophotometer (Thermo Scientific, Waltham, Mass., USA).

Total RNA was extracted from human fibroblast cells and PBMCs from whole blood using RNeasy plus mini kit (Qiagen, Valencia, Calif.) and RIboPure RNA Purification Kit (ThermoFisher Scientific, Waltham, Mass., USA), respectively following manufacturers instruction. RNA quantity and quality was measured by a NanoDrop 2000c Spectrophotometer (Thermo Scientific, Waltham, Mass., USA).

Quantitative RT-PCR.

cDNA was synthesized from mRNA with iScript cDNA Synthesis Kit (Bio-Rad Laboratories, Hercules, Calif., USA) per manufacturer's instruction in a C1000 Touch Thermal Cycler (Bio-Rad Laboratories, Hercules, Calif., USA). A SensiFAST SYBR No-ROX Kit (Bioline, Taunton, Mass., USA) was used to perform qPCR on the synthesized cDNA in a Roche Lightcycler 480 (Roche Diagnostics, Indianapolis, Ind., USA). The second derivative of the amplification curve was used to determine the cycle threshold, and the data were analyzed by a delta delta CT calculation. Primer sets used in qPCR are listed in Table 1.

TABLE 1
Quantitative PCT (qPCR) Primer List

| Species | Gene | Sequence (5′ → 3′) |
|---|---|---|
| Human | MT-TL1 (DNA) | CACCCAAGAACAGGGTTTGT |
| | Forward | |
| | MT-TL1 (DNA) | TGGCCATGGGTATGTTGTTA |
| | Reverse | |
| Human | B2M (DNA) | TGCTGTCTCCATGTTTGATGTATCT |
| | Forward | |
| | B2M (DNA) | TCTCTGCTCCCCACCTCTAAGT |
| | Reverse | |
| Human | TFAM Forward | GTGATTCACCGCAGGAAAAGC |
| | TFAM Reverse | GTGCGACGTAGAAGATCCTTTC |
| Human | NRF1 Forward | AGGAACACGGAGTGACCCAA |
| | NRF1 Reverse | TATGCTCGGTGTAAGTAGCCA |
| Human | NFE2L2 Forward | CAACTACTCCCAGGTTGCCC |
| | NFE2L2 Reverse | AGTGACTGAAACGTAGCCGAA |
| Human | NQO1 Forward | TGGTTTGAGCGAGTGTTCAT |
| | NQO1 Reverse | CCTTCTTACTCCGGAAGGGT |
| Human | HCAR2 Forward | ACAGGTATTTCCGGGTGGTC |
| | HCAR2 Reverse | CGCCATTCTGGATCGGCAT |
| Human | MT-ND2 Forward | CATATACCAAATCTCTCCCTC |
| | MT-ND2 Reverse | GTGCGAGATAGTAGTAGGGTC |
| Human | MT-ND6 Forward | GTAGGATTGGTGCTGTGG |
| | MT-ND6 Reverse | GGATCCTCCCGAATCAAC |
| Human | SDHA Forward | TGCCATCCACTACATGACGG |
| | SDHA Reverse | GCTCTGTCCACCAAATGCAC |
| Human | SDHB Forward | TGGGGCCTGCAGTTCTTATG |
| | SDHB Reverse | ATGGTGTGGCAGCGGTATAG |
| Human | MT-CYB Forward | ACCCCCTAGGAATCACCTCC |
| | MT-CYB Reverse | GCCTAGGAGGTCTGGTGAGA |
| Human | CYC1 Forward | GAGCACGACCATCGAAAACG |
| | CYC1 Reverse | CGATATGCCAGCTTCCGACT |
| Human | MT-CO1 Forward | CGCCGACCGTTGACTATTCT |
| | MT-CO1 Reverse | CGGCTCGAATAAGGAGGCTT |
| Human | MT-CO2 Forward | ACCTTTCATGATCACGCCCT |
| | MT-CO2 Reverse | GGGCAGGATAGTTCAGACGG |
| Human | ATP5B Forward | TGCTCCCATTCATGCTGAGG |
| | ATP5B Reverse | CTCCAGCACCACCAAAAAGC |
| Human | MT-ATP6 Forward | GAAGCGCCACCCTAGCAATA |
| | MT-ATP6 Reverse | GCTTGGATTAAGGCGACAGC |
| Human | β-ACTB Forward | GCCAACACAGTGCTGTCTGG |
| | β-ACTB Reverse | CTGCTTGCTGATCCACATCTGC |
| Mouse | mt-Nd1 (DNA) | TCCGAGCATCTTATCCACGC |
| | Forward | |
| | mt-Nd1 (DNA) | GTATGGTGGTACTCCCGCTG |
| | Reverse | |
| Mouse | Cftr (DNA) | ATGGTCCACAATGAGCCCAG |
| | Forward | |
| | Cftr (DNA) | GAACGAATGACTCTGCCCCT |
| | Reverse | |
| Mouse | mt-Nd2 Forward | ATACTTCGTCACACAAGCAACA |
| | mt-Nd2 Reverse | GGCCTAGTTTTATGGATAGGGCT |
| Mouse | mt-Co1 Forward | CATCTGTTCTGATTCTTTGGGCACC |
| | mt-Co1 Reverse | TGGGCTCATACAATAAAGCCTAGAA |
| Mouse | mt-Atp6 Forward | GCAGTCCGGCTTACAGCTAA |
| | mt-Atp6 Reverse | GGTAGCTGTTGGTGGGCTAA |
| Mouse | Actb Forward | GGCTGTATTCCCCTCCATCG |
| | Actb Reverse | CCAGTTGGTAACAATGCCATGT |

Measurement of Oxygen Consumption in DMF-Treated Fibroblasts by Seahorse XF Analyzer.

Fibroblast cell lines were seeded at a density of 60,000 cells/well in 200 μL of culture medium in a 24-well seahorse tissue culture plate (Seahorse Biosciences, Billerica, Mass., USA). Following a 24-hour incubation, the media was replaced and incubated with 200 μL of 0.1% DMSO or 3 μM, 10 μM and 30 μM dimethyl fumarate in 0.1% DMSO. Prior to reading the oxygen consumption, the medium was changed to unbuffered DMEM without phenol red (Corning, Inc., Corning, N.Y., USA), 10% fetal bovine serum (JR-Scientific, Woodland, Calif., USA), 200 mM glutamax, 100 mM sodium pyruvate, 25 mM glucose (Invitrogen, Waltham, Mass., USA) and was adjusted to pH 7.4. Cells were pre-equilibrated for 20 min; oxygen consumption rate (OCR) and proton production rate (PPR) were recorded with the Seahorse XF-24 after sequential addition of oligomycin (1 ug/ml), FCCP (10 μM) and a combination of antimycin A (1 μM) and rotenone (1 μM) (Sigma-Aldrich, St. Louis, Mo., USA). Total protein in each well was measured and protein concentration was used to normalize the readings.

Data Analysis.

Data analysis was carried out with GraphPad Prism 5.0 statistics software (GraphPad Software, La Jolla, Calif., USA). A list of analysis includes Two-way ANOVA with Bonferroni post-hoc multiple comparison test and One-way ANOVA with Newman-Keuls post-hoc multiple comparison test.

### Results:

DMF Increases Mitochondrial Copy Number, Biogenesis Marker and Subunit Expression, in Human Fibroblasts.

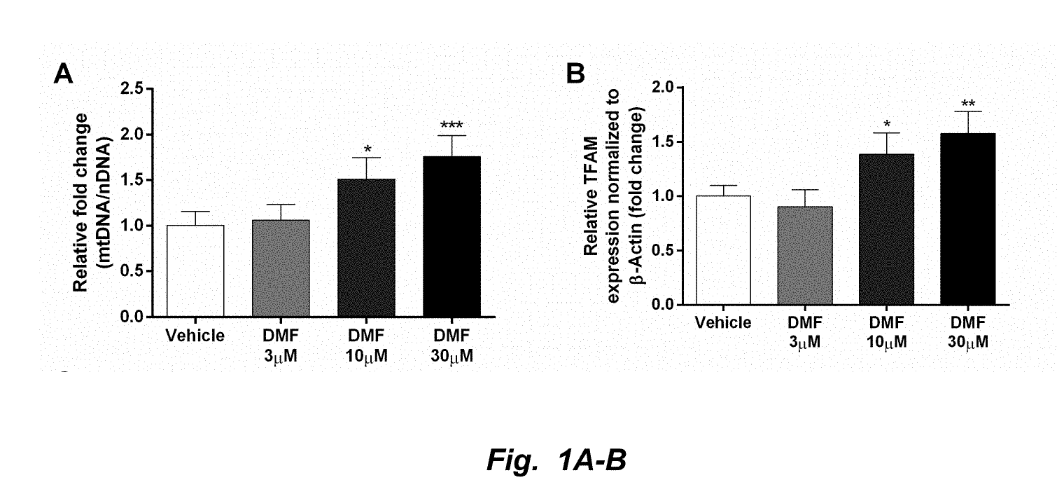

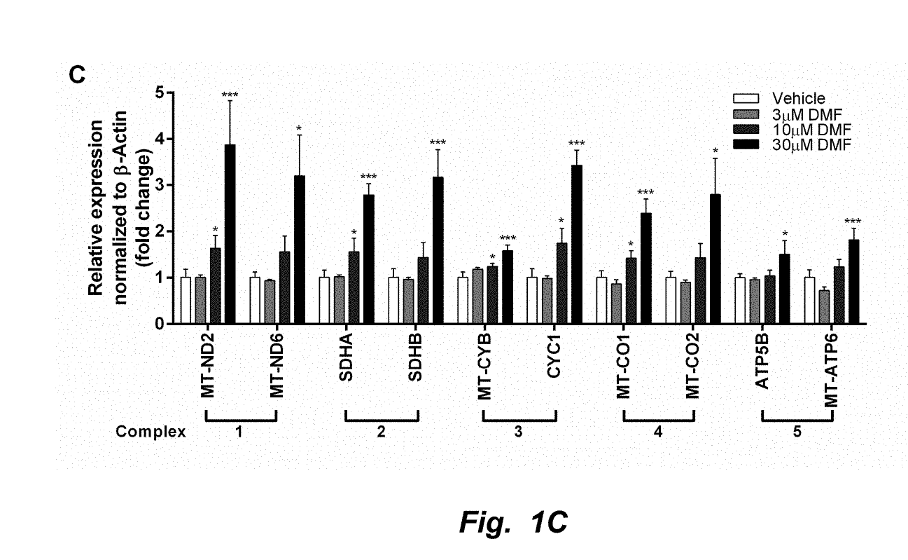

Healthy human fibroblast cells were treated with 0.1% DMSO (vehicle), 3 μM, 10 μM or 30 μM DMF for 48 hours. Mitochondrial DNA (mtDNA) copy number was analyzed from total DNA isolates by measuring the ratio of mitochondrial to nuclear DNA (mtDNA/nDNA). Primers used to amplify mitochondrial DNA and nuclear DNA by qPCR were mitochondrially-encoded tRNA leucine 1 (MT-TL1) and Beta 2 microglobulin (B2M) respectively. While no changes were observed in the 3 M-dosed fibroblasts, 10 μM and 30 μM DMF treatment showed a 1.51 fold (p<0.036, n=3) and 1.75 fold (p<0.0098, n=3) increase in mtDNA copy number compared to vehicle control [FIG. 1A]. Similarly, expression of mitochondrial biogenesis marker TFAM increased at 10 μM and 30 μM DMF when compared to vehicle control [FIG. 1B].

To elucidate whether increased mitochondrial proliferation affects the abundance of the ETC mitochondrial complexes, qPCR analysis was performed to quantify the mRNA expression of numerous complex subunits: complex 1 subunits MT-ND2 and MT-ND6, complex 2 subunits SDHA and SDHB, complex 3 subunits MT-CO1 and MT-CO2, complex 4 subunits MT-CYB and CYC1, and complex 5 subunits ATP5B and MT-ATP6 [Table 1]. Consistent with the DMF-dependent increase in mtDNA copy number and TFAM expression, the subunit expression of the mitochondrial complexes also exhibited a dose-dependent response to DMF treatment. Compared to vehicle treated fibroblasts, average induction of all the complexes were significant when dosed with 10 μM and 30 μM of DMF at 1.42 fold (p<5.7×10⁻⁶, n=10) and 2.65 fold (p<4.8×10⁻⁶, n=10) increase [FIG. 1C]. Interestingly, the expression of complex 5 subunits was less increased in response to DMF treatment compared to other complex subunits, suggesting that there may be a greater need for complexes 1-5 in maintaining a proton gradient necessary for ATP synthesis during mitochondrial biogenesis. Taken together, human fibroblast cells increased mitochondrial biogenesis measured by mtDNA copy number, mitochondrial proliferator marker TFAM expression and mitochondrial complex expression.

Dimethylfumarate Increases Oxygen Consumption Rate in Human Fibroblasts.

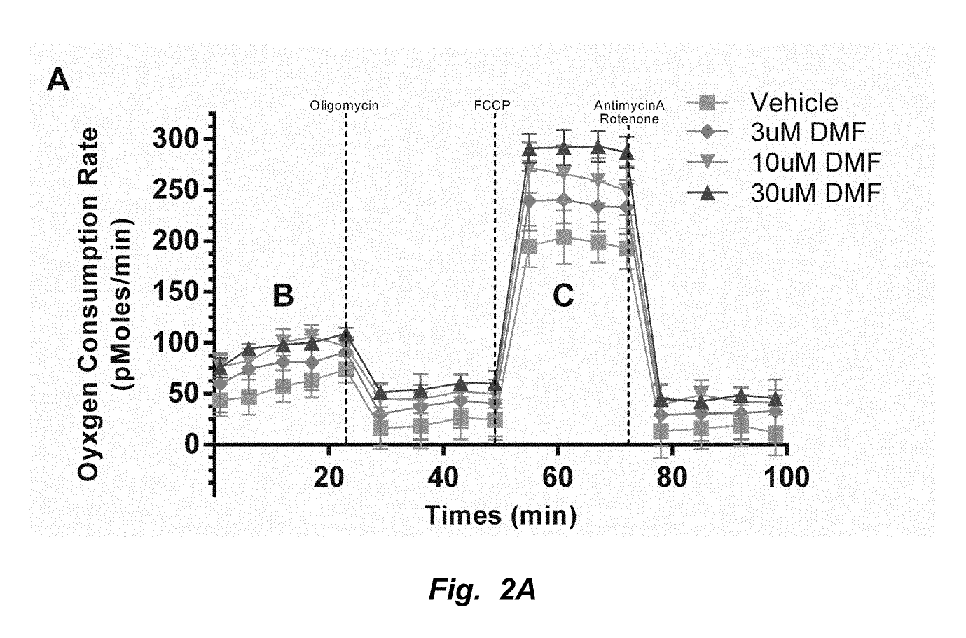

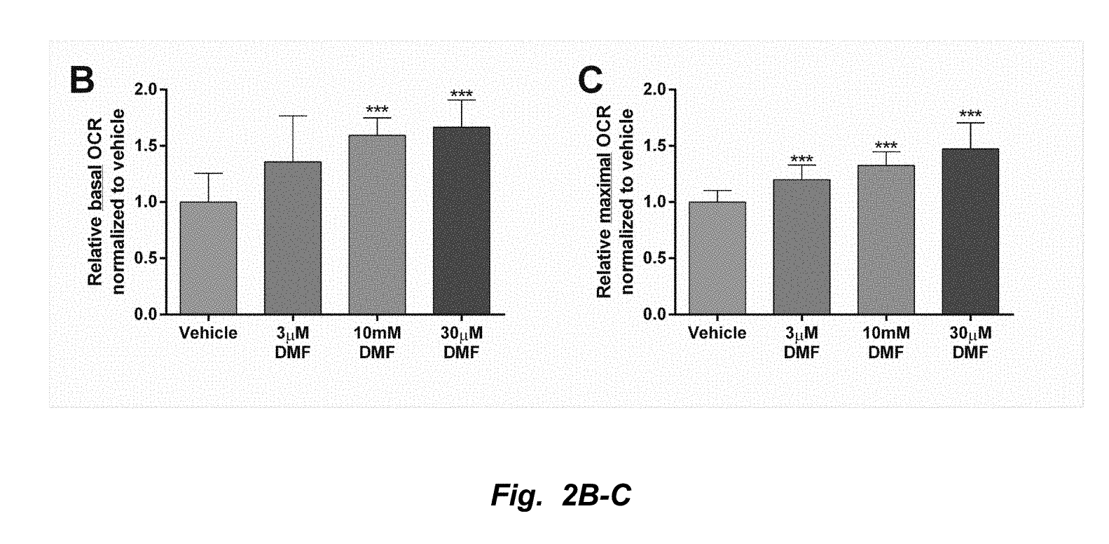

To elucidate the bioenergetic effects of DMF-dependent increases in mitochondrial copy number and mitochondrial complex expression in human fibroblasts, oxygen consumption rate (OCR) was measured. Human fibroblasts were treated with 0.1% DMSO, 3 μM, 10 μM or 30 μM DMF for 48 hours, and oxygen consumption rate (OCR) was sequentially measured in the presence of oligomycin (complex 5 inhibitor), FCCP (mitochondrial uncoupler), and rotenone/antimycin A (Complex 1/3 inhibitor) [FIG. 2A]. The basal OCR reading of fibroblasts treated with 10 μM and 30 μM DMF showed a relative OCR increase of 1.59 fold (p<3.1×10⁻⁵, n=8) and 1.66 fold (p<5.7×10⁻⁵, n=8) compared to vehicle control, respectively [FIG. 2B]. The maximal OCR measured after FCCP injection was elevated after treatment of 3 μM, 10 μM and 30 μM DMF with a relative OCR increase of 1.20 fold (p<3.2×10⁻³, n=8), 1.35 fold (p<3.1×10⁻⁵, n=8), and 1.47 fold (p<9.1×10⁻⁵, n=8) compared to vehicle control, respectively [FIG. 2C]. OCR in the presence of oligomycin and rotenone/antimycin A were not significantly different between the DMF treatment groups and vehicle control [FIG. 2A]. Taken together, human fibroblasts showed DMF dose-dependent induction of basal and maximal (FCCP-treated) OCR at the 48-hour time point. Consistent with the idea that DMF increases mitochondrial copy number and mitochondrial complex expression; the effects of DMF are nullified in the presence of oligomycin and rotenone/antimycin A, which inhibits the mitochondrial electron transport chain that is responsible for mitochondrial oxygen consumption.

Dimethylfumarate Increases Mitochondrial Copy Number and Mitochondrial Complex Expression in Mice.

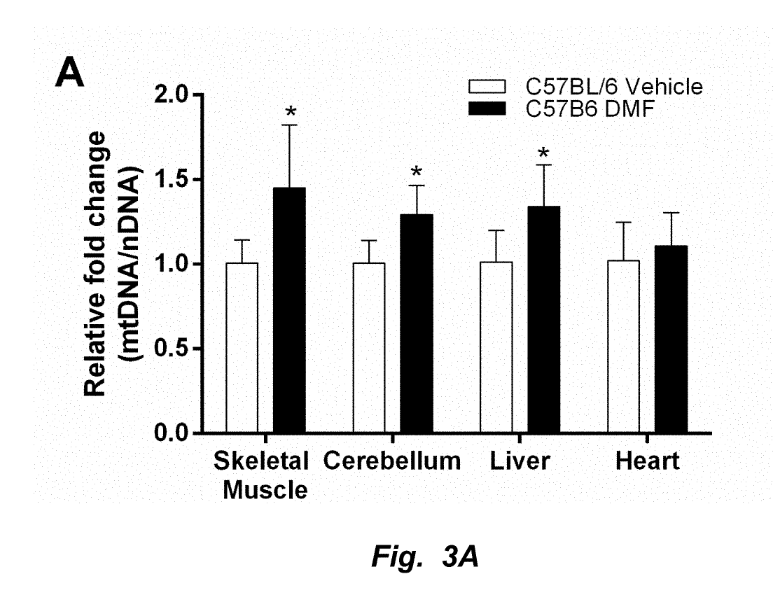

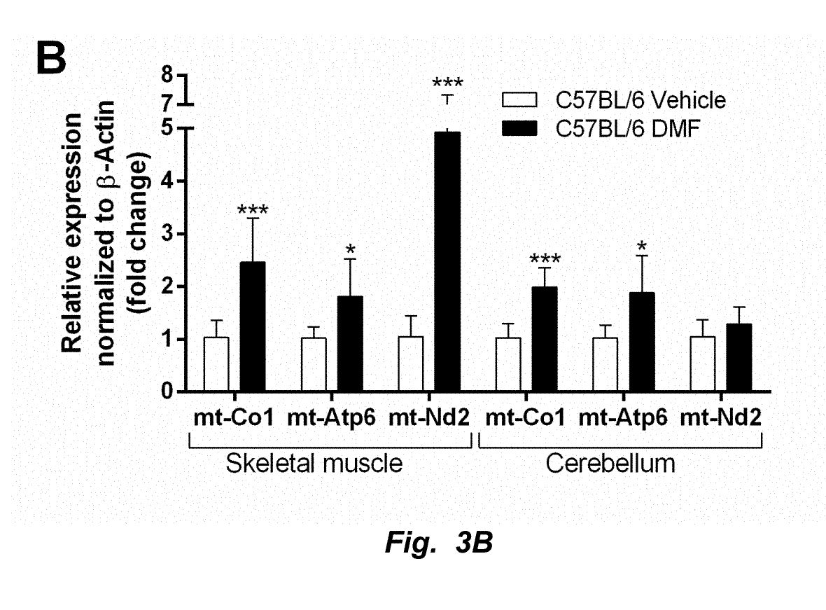

To understand whether the DMF-dependent mitochondrial biogenesis is only applicable to the human fibroblast cells, we examined the effects of DMF on wild type C57BL/6 mice. The mice were dosed with 10 mg/kg DMF daily, and skeletal muscle, cerebellum, liver, and heart tissues were collected after two weeks of treatment We utilized qPCR to analyze, mtDNA copy number as ratio of mt-Nd1 to Cftr and mitochondrial complex expression, in these tissues. Of the four tissues tested, skeletal muscle, cerebellum and liver tissues showed increase in mtDNA copy number by 1.45 fold (p<0.021, n=6), 1.29 fold (p<0.010, n=6) and 1.34 fold (0.027, n=6) respectively. Heart tissue showed no significant change in mtDNA copy number [FIG. 3A]. While DMF seems to induce mitochondrial replication as indicated by mtDNA copy number, it does not seem to affect all tissues equally and suggests that tissue-specific regulation might contribute to this finding.

For the mitochondrial complex expression, skeletal muscle and cerebellar tissue showed significant induction. Both tissues showed an increase in mt-Co1 (complex 4) and mt-Atp6 (complex 5) expression. mt-Co1 and mt-Atp6 was increased 2.38 fold (p<0.0029, n=6) and 1.77 fold (p<0.029, n=6) respectively in skeletal muscle, and 1.92 fold (p<4.5×10⁻⁴, n=6) and 1.82 fold (p<0.020, n=6) respectively in cerebellar tissue. Additionally, skeletal muscle showed 4.68 fold (p<0.0030, n=6) increase in mt-Nd2 expression as a result of two weeks of DMF treatment [FIG. 3B]. Taken together, C57BL/6 mice when dosed with 10 mg/kg of DMF for two weeks show increased mitochondrial copy number and mitochondrial complex expression in multiple tissues. Interestingly, DMF did not increase mtDNA copy in all tissues equally and the expression of some mitochondrial complexes was more induced than others.

Dimethylfumarate Increases Mitochondrial Copy Number and Mitochondrial Complex Expression in MS Patients.

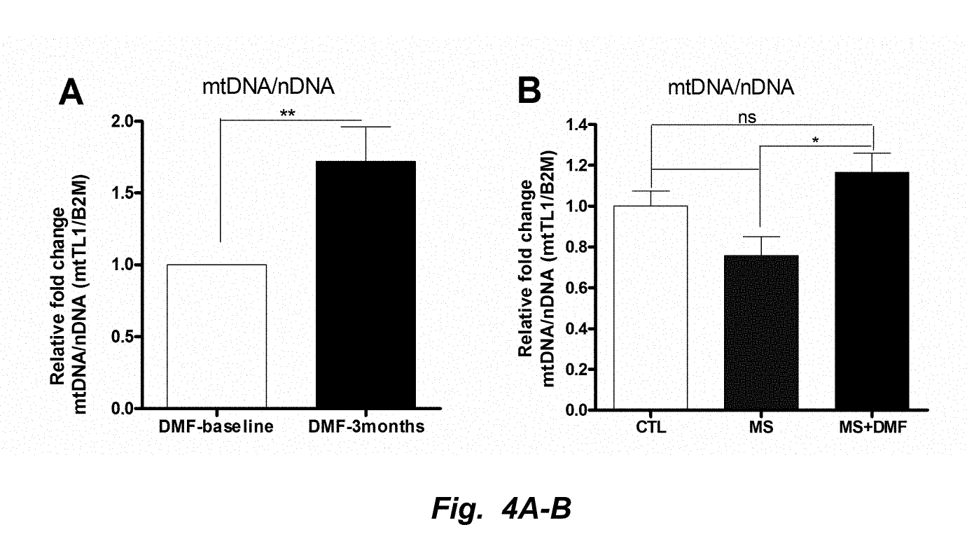

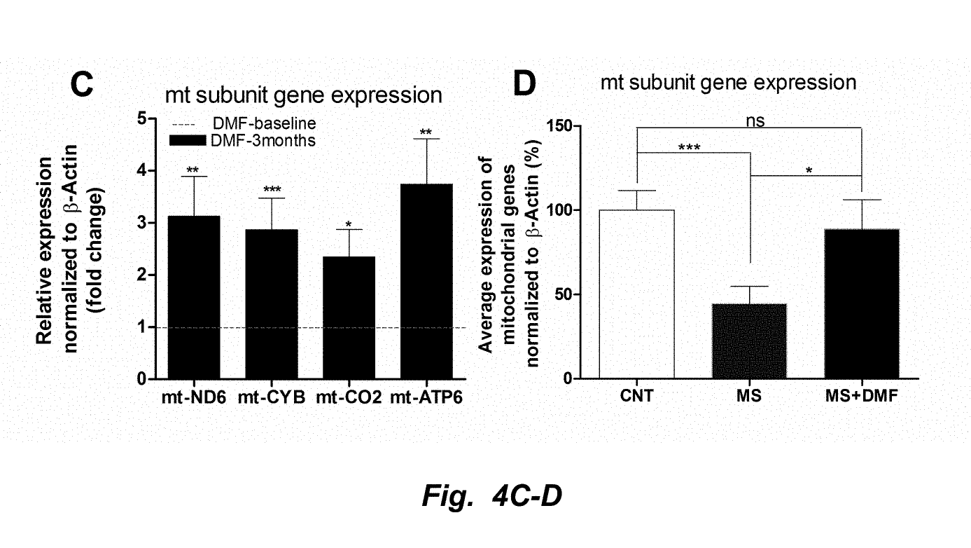

DMF is a FDA approved drug, currently being used to treat adult patients with relapsing form of MS. To confirm whether DMF is contributing to mitochondrial biogenesis when given to human patients, we used qPCR to study mitochondrial DNA copy number and mitochondrial complex subunit expression in peripheral blood lymphocytes isolated from 11 MS patients at baseline and those same patients 3 months after DMF treatment, and 10 controls. We see significant increase in mitochondrial DNA copy number by 71% (p<0.0075, n=11) in MS patients' treated with DMF for 3 months compared to its own baseline [FIG. 4A]. When compared to healthy control group, MS patient at baseline has decreased mitochondrial copy number by 25% (p<0.062, n=11) and 3 month DMF treatment seems to rescue the defect by increasing the mitochondrial DNA copy number back to healthy control level [FIG. 4B].

Similarly, DMF treated MS patients' shows significant increase in mitochondrial complex subunit expression of mt-ND6 (complex 1), mt-CYB (complex 3), mt-CO2 (complex 4) and mt-ATP6 (complex 5) by 3.13 fold (p<0.0358, n=12), 2.87 fold (p<0.016, n=12), 2.34 fold (p<0.041, n=12) and 3.74 fold (p<0.014, n=12) respectively, when normalized to its own baseline. [FIG. 4C]. We also studied the expressions mitochondrial genes: mt-ND6, mt-CYB, mt-CO2 and mt-ATP6 in healthy individuals and compared them to MS patients at baseline and 3 months DMF treatment. We see significant defect in average mitochondrial gene expression in MS patients, a 56% (p<0.0018, n=11) reduction compared to healthy individuals. Following DMF treatment, MS patients showed significant recovery of 88% in average mitochondrial gene expression [FIG. 4D].

DMF's Induction of Mitochondrial Proliferation is Dependent on Nrf2 More than HCAR2.

DMF is known to mediate antioxidant cellular defense by Nrf2 activation, and it suppresses inflammatory signaling by binding to and activating HCAR2 (11, 15). To further understand the Nrf2 and HCAR2 dependent effects of DMF on mitochondrial biogenesis, we analyzed changes in mitochondrial proliferation in Nrf2 siRNA knockdown and HCAR2 siRNA knockdown fibroblasts.

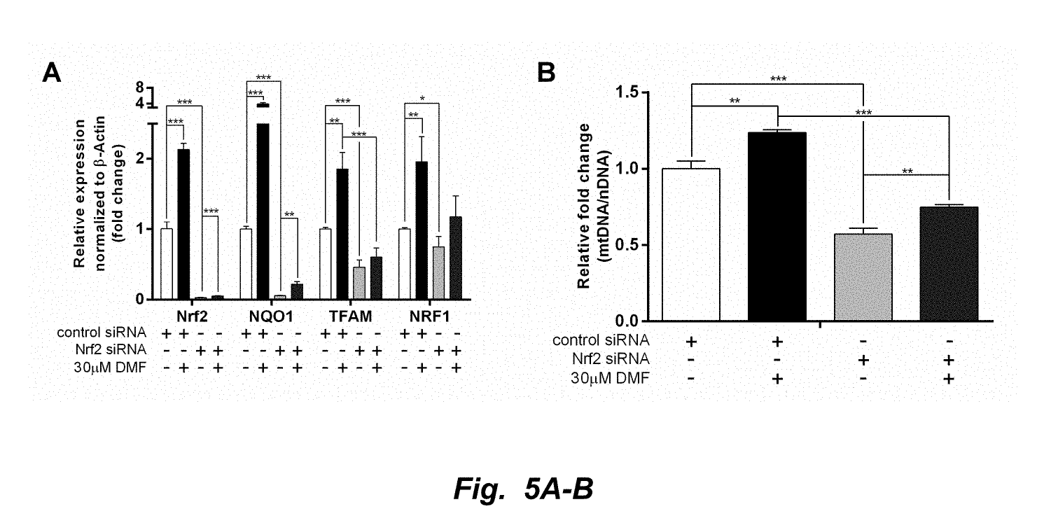

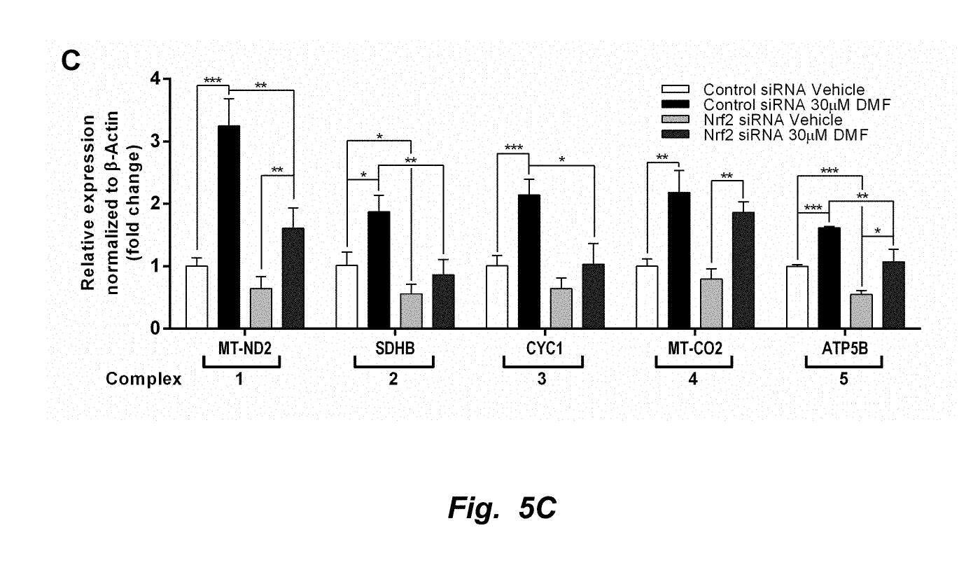

The siNrf2 knockdown significantly reduced Nrf2 expression by 2.5% (p<6.04×10⁻⁵, n=3) of control siRNA treated cells (siCTL). Nrf2 knockdown significantly decreases expression of −NQO1, a downstream target and positive control for Nrf2 activation; TFAM and NRF1, mitochondrial proliferative marker and mt DNA copy number [FIG. 5A,B]. DMF treatment of siCNT cells (siCNT+DMF) showed significant induction of Nrf2, NQO1, TFAM, NRF1 and mtDNA copy number compared to siCNT. DMF treatment of siNrf2 cells (siNrf2+DMF) also significantly increases expression of Nrf2, NQO1 and mtDNA copy number compared to siNrf2 cells. However, the induction of mitochondrial proliferative marker by siNrf2+DMF is significantly reduced as compared to siCTL+DMF. Also, the induction can be attributed to the activation of residual Nrf2 protein. In addition, there was no significant induction of TFAM and NRF1 in siNrf2+DMF. Thus, the results indicate that Nrf2 pathway plays a major role in DMF mediated induction of mitochondrial proliferation.

Quantitative PCR analysis was performed to assess the effect of the Nrf2 pathway on mitochondrial complex subunit expression after DMF treatment. With the exception of mitochondrial complex 4 expressions, siNrf2+DMF cells showed general reduction in mitochondrial complex expression. In addition, siNrf2 basally reduces the expressions of the mitochondrial complexes when vehicle treated [FIG. 5C]. It is apparent that Nrf2 plays a key role in basal and DMF-induced transcription of mitochondrial complex. It is however also important to note that inhibition of DMF induced mitochondrial complexes expression range from slight inhibition in complex 4 indicated by MT-CO2 to large inhibition in complex 2 indicated by SDHB [FIG. 5C]. Taken together, these results indicate dependence of DMF-mediated mitochondrial biogenesis effect on Nrf2 pathway measured by mtDNA copy number and mitochondrial complex expressions.

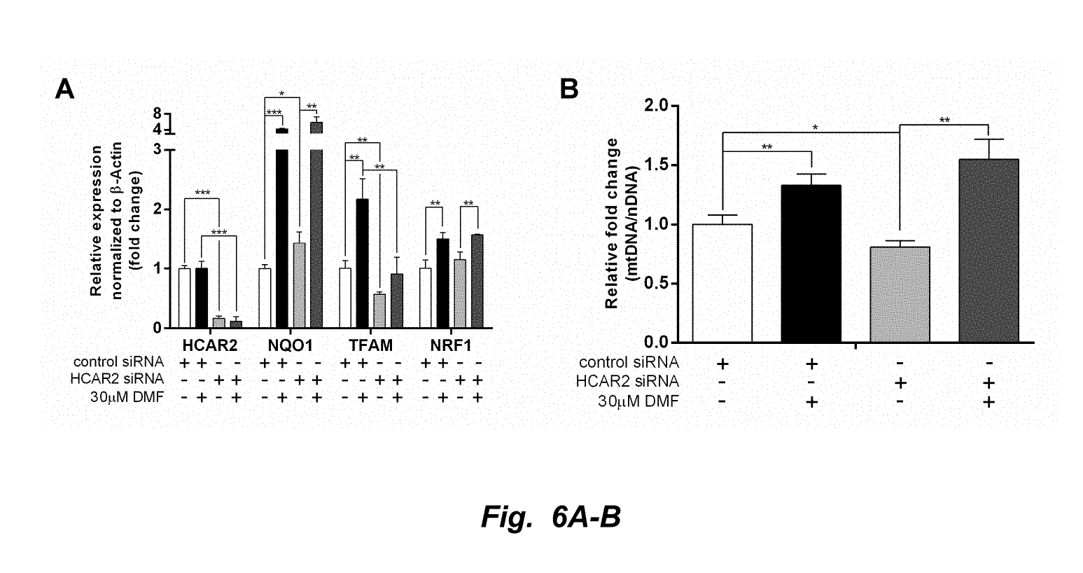

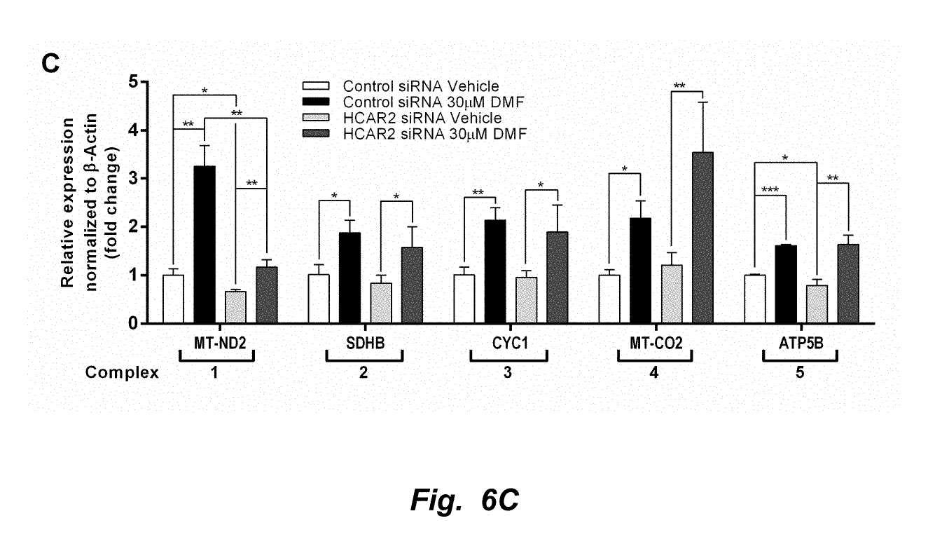

As mentioned earlier, DMF is also known to suppress inflammatory signal by binding to HCAR2 receptor (11, 15). While HCAR2 knockdown significantly reduced HCAR2 expression, DMF treatment of siHCAR2 cells has no significant difference in inducibility of NRF1, mtDNA copy number and mitochondrial complex 2-5 subunit gene expression compared to siCTL+DMF [FIG. 6]. These results indicate that HCAR2 is not involved in DMF mediated mitochondrial proliferation, unlike Nrf2. Interestingly; in HCAR2 knockdown cells there was some effect of DMF treatment on inducibility of complex 1 subunit gene [FIG. 6A-C]. This induction may be due to an alternative pathway discussed below.

### Discussion

Need for Mitochondrial Disease Therapy.

Inherited mitochondrial defects cause serious and lethal disease, for which there is no FDA-approved therapy (2). Identifying pharmacological compounds that can safely and dose-dependently increase mitochondrial copy number and mitochondrial complex expression has potential benefit for those with mitochondrial disease and multiple muscle disease (29, 30). Many muscle diseases depend on mitochondrial function. Muscles contain a paracrystalline formation of mitochondria, whose function is closely tied to overall muscle function. These include not only the mitochondrial myopathies, but also several other muscle dystrophies, including Duchenne dystrophies (31) and ALS (32), which have increasing evidence of mitochondrial involvement. We show here that DMF, an FDA-approved compound, induces mitochondrial biogenesis in healthy human fibroblasts, mouse tissues and humans. DMF dosed systemically in mice clearly produced mitochondrial biogenesis and increased mitochondrial gene expression in muscle, which suggests the potential for ameliorating muscle diseases, which have some mitochondrial pathophysiology as their basis.

Nrf2 and Mitochondrial Biogenesis.

The pharmacological basis of DMF's activity is thought to proceed through its targets Keap1/Nrf2 and the G-protein coupled receptor, HCAR2 (11, 33). While Nrf2 is most commonly known as a major regulator of the antioxidant cellular defense (34), we show that Nrf2 is also necessary for basal mitochondrial maintenance and DMF-induced mitochondrial biogenesis. The transcription factor NRF1 is a key regulator of mitochondrial biogenesis (6-8) with involvements in mitochondrial replication (10) and mtDNA transcription (5, 35). Our data show that basal NRF1 expression was reduced, and its induction by DMF was strikingly diminished in the Nrf2 knockdown line [FIG. 5A]. This observation can be attributed to Nrf2 positive regulation of NRF1 expression by its four ARE motifs (25). Together, the data suggests that DMF activity in part depends on Nrf2-driven NRF1 expression regulating mitochondrial biogenesis. This idea is supported by previous reports showing that Nrf2 binds to the ARE sequence of the NRF1 promoter, inducing mitochondrial biogenesis in cardiomyocytes (25).

In addition to regulating markers of mitochondrial biogenesis, DMF was observed to regulate mitochondrial replication and transcriptions of mitochondrial complexes Nrf2-dependently. We observed a reduction in mtDNA copy number and its reduced inducibility by DMF as a consequence of Nrf2 knock down [FIG. 5B]. Similarly, A previous study by Zhang et al. 2013 showed reduction of mtDNA copy number in the livers of Nrf2 knockout mice (36). The reduction of mtDNA copy as a result of Nrf2 knockdown can be a consequence of diminished mitochondrial proliferation. Additionally, knocking down Nrf2 reduced both basal expression and inducibility of mitochondrial complex subunits expressions by DMF [FIG. 5C]. Similarly, treatments with Nrf2 activators (R)-α-lipoic acid and acetyl-L-carnitine can promote mitochondrial proliferation and function in adipocytes (26). Taken together, induction of mitochondrial replication and mitochondrial complex expressions by DMF is dependent on Nrf2 pathway.

Aside from promoters and binding, there is also the physiological question of why Nrf2 and mitochondrial biogenesis pathways are co-regulated in both positive and negative directions. Positively, one could imagine that increased fat content in the diet and agonism of the HCAR2 beta-hydroxybutyrate receptor could signal increased mitochondrial biogenesis in order to carry out more active oxidative phosphorylation on the more reduced fatty foodstuff, which in turn would presumably produce more ROS and thus require more Nrf2. In the negative direction, a suppression of Nrf2 results in a decreased antioxidant response, and cells may protect themselves by decreasing mitochondrial number, as mitochondria are a major contributor to ROS production.

DMF's Tissue-Specific Effects.

Similar to the in vitro responses seen in fibroblast cells, two-week intraperitoneal DMF dosing of C57BL/6 mice showed increase in mtDNA copy number and mitochondrial complex expression in vivo [FIG. 3]. Three of the four tissues tested: skeletal muscle (gastrocnemius), whole cerebellum, and whole liver, were observed to have increased mtDNA in DMF-treated mice as compared to vehicle-treated mice [FIG. 3A]. We additionally observed induction of mitochondrial complexes in the skeletal muscle and cerebellum [FIG. 3B], effects in liver or heart tissues were more minor. DMF's tissue-specific effects on mitochondrial gene expression could result from differential expression of its two known targets Nrf2 (37-40) and HCAR2 (41-43).

Contribution of HCAR2 to DMF's Effects on Mitochondrial Complex I.

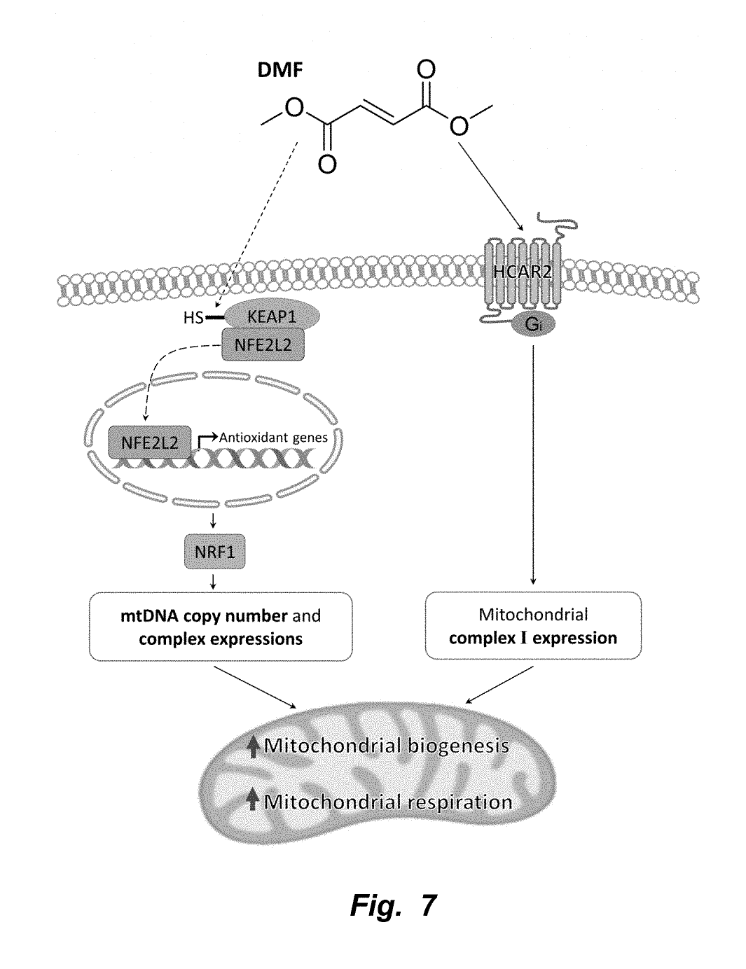

Of the two DMF targets, Nrf2 and HCAR2, our results suggest that HCAR2 plays a smaller role in the mitochondrial biogenesis effect of DMF, except for some small effects on mitochondrial complex 1, or additive effects. When HCAR2 expression is knocked down, complex 1 subunit MT-ND2 became uninducible by DMF [FIG. 6]. HCAR2 is also known as the NIACR1 (niacin receptor), and thus can detect the bioavailability of nicotinic acid (44), a precursor to nicotinic dinucleotide (NAD+) (45), the major redox substrate of mitochondrial complex 1(1). Thus HCAR2 knockdown could reduce the signaling of DMF and other HCAR2 agonists such as niacin in a feedforward stimulation of complex 1. But besides complex 1, knockdown of HCAR2 did not affect DMF-dependent mitochondrial gene expression in a major way [FIG. 6]. It is possible that there is some synergy among DMF's Nrf2 and HCAR2-dependent mitochondrial effects, with Nrf2 the main player but HCAR2 playing an additive role [FIG. 7].

Mitochondrial Gene Expression as Blood Based Biomarker for MS.

Mitochondrial dysfunction is considered one of the several causes of axonal neuron degradation in MS (46). Ours is the first reported demonstration that mitochondrial copy number and mitochondrial gene expression is decreased in MS blood lymphocytes relative to healthy controls and the observed defect is ameliorated by DMF treatment in MS patients [FIG. 4]. These results suggest that mitochondrial copy number and mitochondrial gene expression can be used as a potential biomarker in the neurodegenerative disease MS, for example as a biomarker of disease severity, disease form (progressive or relapsing-remitting), and response to treatment. Consistent with the cell and mouse model experiments, DMF-dosed MS patients had increased mitochondrial copy number and gene expression in blood lymphocytes. [FIG. 4]. Thus, DMF treatment increases mitochondrial gene expression in both mice and humans, and was sufficient to rescue the mitochondrial gene expression deficit in MS patients.

Prospects for DMF as a Drug for Mitochondrial Biogenesis and Muscle Disease.

The findings reported here demonstrate novel mitochondrial proliferative pharmacological properties of the FDA-approved drug dimethyl fumarate, which appear to depend mainly on the Nrf2 pathway [FIG. 6]. DMF increases mtDNA copy number [FIGS. 1A and 3A] and mitochondrial complex mRNA expression [FIGS. 1C and 3B] in human fibroblasts and WT mice, and rescues a mitochondrial deficit in MS patients [FIG. 4]. In contrast to other potential mitoproliferative compounds not clinically available, DMF is an approved drug in US and Europe with demonstrated mitoproliferative activity. DMF is currently used to treat psoriasis, an autoimmune disease, and Multiple Sclerosis, a demyelinating disease (13). In the past, the drug pioglitazone, a thiazolidinedione used to treat diabetic patients, and bezafibrate (2) were shown to have mitochondrial proliferative effect. Pioglitazone was later shown to inhibit mitochondrial complex I (47). DMF on the other hand does not appear to have mitochondrial complex inhibition activity as indicated by induction of both basal and maximal respiration [FIG. 2] while simultaneously increasing mitochondrial DNA copy number and mitochondrial gene expression [FIG. 1]. This provides a greater confidence that FDA approved drug DMF could be considered for diseases in which there is reduced mitochondrial function including mitochondrial and muscle disease could support mitochondrial proliferation and health.

### REFERENCES

1. Schapira A H. Mitochondrial disease. Lancet (London, England). 2006; 368(9529):70-82.
2. Wenz T, Diaz F, Spiegelman B M, and Moraes C T. Activation of the PPAR/PGC-1 alpha pathway prevents a bioenergetic deficit and effectively improves a mitochondrial myopathy phenotype. Cell metabolism. 2008; 8(3):249-56.
3. Puigserver P, Wu Z, Park C W, Graves R, Wright M, and Spiegelman B M. A cold-inducible coactivator of nuclear receptors linked to adaptive thermogenesis. Cell. 1998; 92(6): 829-39.
4. Virbasius J V, and Scarpulla R C. Activation of the human mitochondrial transcription factor A gene by nuclear respiratory factors: a potential regulatory link between nuclear and mitochondrial gene expression in organelle biogenesis. Proceedings of the National Academy of Sciences of the United States of America 1994; 91(4):1309-13.
5. Gugneja S, Virbasius C M, and Scarpulla R C. Nuclear respiratory factors 1 and 2 utilize similar glutamine-containing clusters of hydrophobic residues to activate transcription. Molecular and cellular biology. 1996; 16(10):5708-16.
6. Evans M J, and Scarpulla R C. NRF-1: a trans-activator of nuclear-encoded respiratory genes in animal cells. Genes & development. 1990; 4(6):1023-34.
7. Virbasius C A, Virbasius J V, and Scarpulla R C. NRF-1, an activator involved in nuclear-mitochondrial interactions, utilizes a new DNA-binding domain conserved in a family of developmental regulators. Genes & development. 1993; 7(12a):2431-45.
8. Wu Z, Puigserver P, Andersson U, Zhang C, Adelmant G, Mootha V, Troy A, Cinti S, Lowell B, Scarpulla R C, et al. Mechanisms controlling mitochondrial biogenesis and respiration through the thermogenic coactivator PGC-1. Cell. 1999; 98(1):115-24.
9. Gleyzer N, Vercauteren K, and Scarpulla R C. Control of mitochondrial transcription specificity factors (TFB1M and TFB2M) by nuclear respiratory factors (NRF-1 and NRF-2) and PGC-1 family coactivators. Molecular and cellular biology. 2005; 25(4):1354-66.
10. Ekstrand M I, Falkenberg M, Rantanen A, Park C B, Gaspari M, Hultenby K, Rustin P, Gustafsson C M, and Larsson N G. Mitochondrial transcription factor A regulates mtDNA copy number in mammals. Human molecular genetics. 2004; 13(9):935-44.
11. Linker R A, Lee D H, Ryan S, van Dam A M, Conrad R, Bista P, Zeng W, Hronowsky X, Buko A, Chollate S, et al. Fumaric acid esters exert neuroprotective effects in neuroinflammation via activation of the Nrf2 antioxidant pathway. Brain: a journal of neurology. 2011; 134(Pt 3):678-92.
12. Scannevin R H, Chollate S, Jung M Y, Shackett M, Patel H, Bista P, Zeng W, Ryan S, Yamamoto M, Lukashev M, et al. Fumarates promote cytoprotection of central nervous system cells against oxidative stress via the nuclear factor (erythroid-derived 2)-like 2 pathway. The Journal of pharmacology and experimental therapeutics. 2012; 341(1):274-84.
13. Fox R J, Miller D H, Phillips J T, Hutchinson M, Havrdova E, Kita M, Yang M, Raghupathi K, Novas M, Sweetser M T, et al. Placebo-controlled phase 3 study of oral BG-12 or glatiramer in multiple sclerosis. The New England journal of medicine. 2012; 367(12):1087-97.
14. Mrowietz U, Altmeyer P, Bieber T, Rocken M, Schopf R E, and Sterry W. Treatment of psoriasis with fumaric acid esters (Fumaderm). Journal der Deutschen Dermatologischen Gesellschaft=Journal of the German Society of Dermatology: JDDG. 2007; 5(8):716-7.
15. Chen H, Assmann J C, Krenz A, Rahman M, Grimm M, Karsten C M, Kohl J, Offermanns S, Wettschureck N, and Schwaninger M. Hydroxycarboxylic acid receptor 2 mediates dimethyl fumarate's protective effect in EAE. The Journal of clinical investigation. 2014; 124(5):2188-92.
16. Wild A C, Moinova H R, and Mulcahy R T. Regulation of gamma-glutamylcysteine synthetase subunit gene expression by the transcription factor Nrf2. The Journal of biological chemistry. 1999; 274(47):33627-36.
17. Harvey C J, Thimmulappa R K, Singh A, Blake D J, Ling G, Wakabayashi N, Fujii J, Myers A, and Biswal S. Nrf2-regulated glutathione recycling independent of biosynthesis is critical for cell survival during oxidative stress. Free radical biology & medicine. 2009; 46(4):443-53.
18. Kensler T W, Wakabayashi N, and Biswal S. Cell survival responses to environmental stresses via the Keapl-Nrf2-ARE pathway. Annual review of pharmacology and toxicology. 2007; 47(89-116.
19. Calkins M J, Johnson D A, Townsend J A, Vargas M R, Dowell J A, Williamson T P, Kraft A D, Lee J M, Li J, and Johnson J A. The Nrf2/ARE pathway as a potential therapeutic target in neurodegenerative disease. Antioxidants & redox signaling. 2009; 11(3):497-508.
20. Dinkova-Kostova A T, and Abramov A Y. The emerging role of Nrf2 in mitochondrial function. Free Radical Biology and Medicine. 2015; 88, Part B (179-88.
21. Brennan M S, Matos M F, Li B, Hronowski X, Gao B, Juhasz P, Rhodes K J, and Scannevin R H. Dimethyl fumarate and monoethyl fumarate exhibit differential effects on KEAP1, NRF2 activation, and glutathione depletion in vitro. PloS one. 2015; 10(3):e0120254.
22. Yamamoto T, Suzuki T, Kobayashi A, Wakabayashi J, Maher J, Motohashi H, and Yamamoto M. Physiological significance of reactive cysteine residues of Keapl in determining Nrf2 activity. Molecular and cellular biology. 2008; 28(8):2758-70.
23. Nguyen T, Sherratt P J, Nioi P, Yang C S, and Pickett C B. Nrf2 controls constitutive and inducible expression of ARE-driven genes through a dynamic pathway involving nucleocytoplasmic shuttling by Keap1. The Journal of biological chemistry. 2005; 280(37):32485-92.
24. Dinkova-Kostova A T, and Abramov A Y. The emerging role of Nrf2 in mitochondrial function. Free Radic Biol Med. 2015; 88(Pt B):179-88.
25. Piantadosi C A, Carraway M S, Babiker A, and Suliman H B. Heme oxygenase-1 regulates cardiac mitochondrial biogenesis via Nrf2-mediated transcriptional control of nuclear respiratory factor-1. Circulation research. 2008; 103(11):1232-40.
26. Shen W, Liu K, Tian C, Yang L, Li X, Ren J, Packer L, Cotman C W, and Liu J. R-alpha-lipoic acid and acetyl-L-carnitine complementarily promote mitochondrial biogenesis in murine 3T3-L1 adipocytes. Diabetologia. 2008; 51(1):165-74.
27. Tang H, Lu J Y, Zheng X, Yang Y, and Reagan J D. The psoriasis drug monomethylfumarate is a potent nicotinic acid receptor agonist. Biochemical and biophysical research communications. 2008; 375(4):562-5.
28. Sahdeo S, Tomilov A, Komachi K, Iwahashi C, Datta S, Hughes O, Hagerman P, and Cortopassi G. High-throughput screening of FDA-approved drugs using oxygen biosensor plates reveals secondary mitofunctional effects. Mitochondrion. 2014; 17(116-25.
29. Bhat A H, Dar K B, Anees S, Zargar M A, Masood A, Sofi M A, and Ganie S A. Oxidative stress, mitochondrial dysfunction and neurodegenerative diseases; a mechanistic insight. Biomedicine & pharmacotherapy=Biomedecine & pharmacotherapie. 2015; 74(101-10.
30. Katsetos C D, Koutzaki S, and Melvin J J. Mitochondrial dysfunction in neuromuscular disorders. Seminars in pediatric neurology. 2013; 20(3):202-15.
31. Percival J M, Siegel M P, Knowels G, and Marcinek D J. Defects in mitochondrial localization and ATP synthesis in the mdx mouse model of Duchenne muscular dystrophy are not alleviated by PDE5 inhibition. Human molecular genetics. 2013; 22(1):153-67.
32. Cozzolino M, and Carri M T. Mitochondrial dysfunction in ALS. Progress in neurobiology. 2012; 97(2):54-66.
33. Parodi B, Rossi S, Morando S, Cordano C, Bragoni A, Motta C, Usai C, Wipke B T, Scannevin R H, Mancardi G L, et al. Fumarates modulate microglia activation through a novel HCAR2 signaling pathway and rescue synaptic dysregulation in inflamed CNS. Acta neuropathologica. 2015; 130(2):279-95.
34. Ma Q. Role of nrf2 in oxidative stress and toxicity. Annual review of pharmacology and toxicology. 2013; 53(401-26.
35. Fisher R P, and Clayton D A. Purification and characterization of human mitochondrial transcription factor 1. Molecular and cellular biology. 1988; 8(8):3496-509.
36. Zhang Y K, Wu K C, and Klaassen C D. Genetic activation of Nrf2 protects against fasting-induced oxidative stress in livers of mice. PloS one. 2013; 8(3):e59122.
37. Cho H Y, Reddy S P, Yamamoto M, and Kleeberger S R The transcription factor NRF2 protects against pulmonary fibrosis. FASEB joumal: official publication of the Federation of American Societies for Experimental Biology. 2004; 18(11): 1258-60.
38. Li J, Stein T D, and Johnson J A. Genetic dissection of systemic autoimmune disease in Nrf2-deficient mice. Physiological genomics. 2004; 18(3):261-72.
39. Lee D H, Gold R, and Linker R A. Mechanisms of Oxidative Damage in Multiple Sclerosis and Neurodegenerative Diseases: Therapeutic Modulation via Fumaric Acid Esters. International journal of molecular sciences. 2012; 13(9): 11783-803.
40. Al-Sawaf O, Fragoulis A, Rosen C, Keimes N, Liehn E A, Holzle F, Kan Y W, Pufe T, Sonmez T T, and Wruck C J. Nrf2 augments skeletal muscle regeneration after ischaemia-reperfusion injury. The Journal of pathology. 2014; 234(4):538-47.
41. Li X, Millar J S, Brownell N, Briand F, and Rader D J. Modulation of HDL metabolism by the niacin receptor GPR109A in mouse hepatocytes. Biochemical pharmacology. 2010; 80(9):1450-7.
42. Rahman M, Muhammad S, Khan M A, Chen H, Ridder D A, Muller-Fielitz H, Pokoma B, Vollbrandt T, Stolting I, Nadrowitz R, et al. The beta-hydroxybutyrate receptor HCA2 activates a neuroprotective subset of macrophages. Nature communications. 2014; 5(3944.
43. Couturier A, Ringseis R, Most E, and Eder K Pharmacological doses of niacin stimulate the expression of genes involved in camitine uptake and biosynthesis and improve the camitine status of obese Zucker rats. BMC pharmacology & toxicology. 2014; 15(37.
44. Soga T, Kamohara M, Takasaki J, Matsumoto S, Saito T, Ohishi T, Hiyama H, Matsuo A, Matsushime H, and Furuichi K. Molecular identification of nicotinic acid receptor. Biochemical and biophysical research communications. 2003; 303(1):364-9.
45. Hara N, Yamada K, Shibata T, Osago H, Hashimoto T, and Tsuchiya M. Elevation of cellular NAD levels by nicotinic acid and involvement of nicotinic acid phosphoribosyltransferase in human cells. The Journal of biological chemistry. 2007; 282(34):24574-82.
46. Mao P, and Reddy P H. Is multiple sclerosis a mitochondrial disease? Biochimica et biophysica acta 2010; 1802(1):66-79.
47. Garcia-Ruiz I, Solis-Munoz P, Femandez-Moreira D, Munoz-Yague T, and Solis-Herruzo J A. Pioglitazone leads to an inactivation and disassembly of complex I of the mitochondrial respiratory chain. BMC biology. 2013; 11(88.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of promoting and/or increasing mitochondrial mass and/or functionality in a mammalian myocyte and/or hepatocyte, comprising contacting the myocyte and/or hepatocyte with a compound of Formula (I) or a pharmaceutically acceptable salt thereof; wherein R¹ and R² are independently selected from —CH₃, —OH, —O, -E, and C1-C8 alkoxy (branched or unbranched), provided that at least one of R¹ and R² is C1-C8 alkoxy: 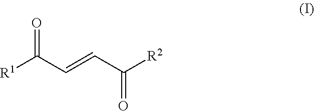
under conditions sufficient to increase mitochondrial mass and/or functionality in a mammalian myocyte and/or hepatocyte.

2. The method of claim 1, wherein the compound of Formula (I) comprises a fumarate ester.

3. The method of claim 1, wherein the compound of Formula (I) is selected from the group consisting of monomethyl fumarate (MMF), monomethyl maleate, monoethyl fumarate, monoethyl maleate, monobutyl fumarate, monobutyl maleate, monooctyl fumarate, monoctyl maleate, mono (phenylmethyl) fumarate, mono (phenylmethyl) maleate, mono (2-hydroxypropyl) fumarate, mono (2-hydroxypropyl) maleate, mono (2-ethylhexyl) fumarate, mono (2-ethylhexyl) maleate, dimethylfumarate, dimethyl maleate, diethyl fumarate, diethyl maleate, dipropyl fumarate, dipropyl maleate, diisopropyl fumarate, diisopropyl maleate, dibutyl fumarate, dibutyl maleate, diisobutyl fumarate, diisobutyl maleate, diheptyl fumarate, diheptyl maleate, bis (2-ethylhexyl) fumarate, bis (2-ethylhexyl) maleate, (−)-Dimenthyl fumarate, (−)-Bis ((S)-1-(ethoxycarbonyl)ethyl) fumarate, (−)-Bis ((S)-1-(ethoxycarbonyl)ethyl) maleate, Bis (2-trifluoroethyl) fumarate, Bis (2-trifluoroethyl) maleate, and mixtures thereof.

4. The method of claim 1, wherein the compound of Formula (I) comprises dimethyl fumarate (DMF).

5. The method of claim 1, further comprising contacting the myocyte and/or hepatocyte with methylene blue.

6. (canceled)

7. The method of claim 1, wherein the mitochondrial mass is increased by at least about 25%.

8. The method of claim 1, wherein the mitochondrial copy number/nucleus is increased by at least about 100%.

9. The method of claim 1, wherein the myocyte and/or hepatocyte is contacted with the compound of Formula (I) at a concentration in the range of about 1 μM to about 50 μM.

10. The method of claim 1, wherein the compound of Formula (I) is formulated in a cyclodextrin.

11. The method of claim 10, wherein the cyclodextrin is selected from the group consisting of hydroxypropyl-β-cyclodextrin, endotoxin controlled β-cyclodextrin sulfobutyl ethers, or cyclodextrin sodium salts.

12. The method of claim 1, wherein the myocyte and/or hepatocyte is human.

13. The method of claim 1, wherein the myocyte and/or hepatocyte is in vitro.

14. The method of claim 1, wherein the myocyte and/or hepatocyte is in vivo.

15. The method of claim 1, wherein the myocyte is a skeletal myocyte or a cardiomyocyte.

16. The method of claim 14, wherein the myocyte is in or from a subject suffering from a muscle disorder.

17. The method of claim 16, wherein the muscle disorder involves muscle wasting.

18. The method of claim 16, wherein the muscle disorder is selected from the group consisting of Cancer cachexia, age-related muscle wasting (sarcopenia), Mitochondrial myopathy, Acid Maltase Deficiency (AMD), Amyotrophic Lateral Sclerosis (ALS), Amyotrophy, Andersen-Tawil Syndrome, Anterior compartment syndrome of the lower leg, Becker Muscular Dystrophy (BMD), Becker Myotonia Congenita, Bethlem Myopathy, Bimagrumab, Bulbospinal Muscular Atrophy (Spinal-Bulbar Muscular Atrophy), Carnitine Deficiency, Carnitine Palmityl Transferase Deficiency (CPT Deficiency), Cataplexy, Central core disease of muscle, Centronuclear Myopathy, Charcot-Marie-Tooth Disease (CMT), Charley horse, Chronic fatigue syndrome, Chronic progressive external ophthalmoplegia, Congenital Muscular Dystrophy (CMD), Congenital Myasthenic Syndromes (CMS), Congenital Myotonic Dystrophy, Contracture, Cori Disease (Debrancher Enzyme Deficiency), Cramp, Cricopharyngeal spasm, Debrancher Enzyme Deficiency, Dejerine-Sottas Disease (DSD), Dermatomyositis (DM), Diastasis recti, Distal Muscular Dystrophy (DD), Distal spinal muscular atrophy type 2, Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Exercise therapy for idiopathic inflammatory myopathies, Exercise-associated muscle cramps, Exertional rhabdomyolysis, Facioscapulohumeral Muscular Dystrophy (FSH or FSHD), Fibrodysplasia ossificans progressive, Finnish (Tibial) Distal Myopathy, Forbes Disease (Debrancher Enzyme Deficiency), Fukuyama Congenital Muscular Dystrophy, Glycogen storage disease type XI, Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery-Dreifuss Muscular Dystrophy), Hereditary inclusion body myopathy and myositis, Hereditary Motor and Sensory Neuropathy (Charcot-Marie-Tooth Disease), Hyperthyroid Myopathy, Hypertonia, Hypothyroid Myopathy, Inclusion-Body Myositis (IBM) and myopathy, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Laminopathy, Late-onset mitochondrial myopathy, Limb-Girdle Muscular Dystrophy (LGMD), Lou Gehrig's Disease (Amyotrophic Lateral Sclerosis), Macrophagic myofasciitis, McArdle Disease (Phosphorylase Deficiency), Merosin-Deficient Congenital Muscular Dystrophy, Metabolic myopathy, Mitochondrial Myopathy, Miyoshi Distal Myopathy, Motor Neurone Disease, Muscle atrophy, Muscle fatigue, Muscle imbalance, Muscle weakness, Muscle-Eye-Brain Disease, Myasthenia Gravis (MG), Myoadenylate Deaminase Deficiency, Myofibrillar Myopathy, Myopathy, Myopathy, X-linked, with excessive autophagy, Myophosphorylase Deficiency, Myositis, Myositis ossificans, Myostatin-related muscle hypertrophy, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Orofacial myological disorders, Paramyotonia Congenita, Paratonia, Pearson Syndrome, Pelvic floor muscle disorder, Periodic Paralysis, Peroneal Muscular Atrophy (Charcot-Marie-Tooth Disease), Phosphofructokinase Deficiency, Phosphoglycerate Kinase Deficiency, Phosphorylase Deficiency, Polymyositis (PM), Pompe Disease (Acid Maltase Deficiency), Progressive External Ophthalmoplegia (PEO), Psoas muscle abscess, Pyomyositis, Rod Body Disease (Nemaline Myopathy), Sarcoglycanopathy, Sphincter paralysis, Spinal Muscular Atrophy (SMA), Spinal-Bulbar Muscular Atrophy (SBMA)/Kennedy's disease, Steinert Disease (Myotonic Muscular Dystrophy), Strain (injury), Tarui Disease (Phosphofructokinase Deficiency), Thomsen Disease (Myotonia Congenita), Thyrotoxic periodic paralysis, Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), ZASP-Related Myopathy and Zenker's degeneration.

19. The method of claim 16, wherein the muscle disorder is a muscular dystrophy.

20. The method of claim 14, wherein the hepatocyte is in or from a subject suffering from a liver disorder.

21. The method of claim 20, wherein the liver disorder is selected from the group consisting of mitochondrial liver disease, hepatitis, alcoholic liver disease, fatty liver disease (hepatic steatosis), NASH-Non-alcoholic steatohepatitis, Gilbert's syndrome, cirrhosis, primary liver cancer, primary biliary cirrhosis, primary sclerosing cholangitis, and Budd-Chiari syndrome.

22. A method of promoting and/or increasing mitochondrial mass and/or functionality in the muscle tissue and/or liver tissue in a subject in need thereof comprising administering to the subject a therapeutically effective regime of a compound of Formula (I) or a pharmaceutically acceptable salt thereof; wherein R¹ and R² are independently selected from —CH₃, —OH, —O, -E, and C1-C8 alkoxy (branched or unbranched), provided that at least one of R¹ and R² is C1-C8 alkoxy:

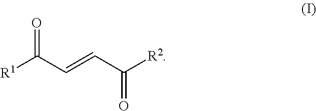

23. A method of preventing, delaying, reducing, mitigating, ameliorating and/or inhibiting one or more symptoms associated with a muscle disorder or a liver disorder in a subject in need thereof comprising administering to the subject a therapeutically effective regime of a compound of Formula (I) or a pharmaceutically acceptable salt thereof; wherein R¹ and R² are independently selected from —CH₃, —OH, —O, -E, and C1-C8 alkoxy (branched or unbranched), provided that at least one of R¹ and R² is C1-C8 alkoxy:

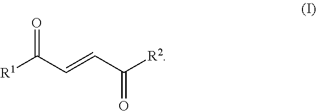

24. The method of claim 22, wherein the compound of Formula (I) comprises a fumarate ester.

25. The method of claim 22, wherein the compound of Formula (I) is selected from the group consisting of monomethyl fumarate (MMF), monomethyl maleate, monoethyl fumarate, monoethyl maleate, monobutyl fumarate, monobutyl maleate, monooctyl fumarate, monoctyl maleate, mono (phenylmethyl) fumarate, mono (phenylmethyl) maleate, mono (2-hydroxypropyl) fumarate, mono (2-hydroxypropyl) maleate, mono (2-ethylhexyl) fumarate, mono (2-ethylhexyl) maleate, dimethylfumarate, dimethyl maleate, diethyl fumarate, diethyl maleate, dipropyl fumarate, dipropyl maleate, diisopropyl fumarate, diisopropyl maleate, dibutyl fumarate, dibutyl maleate, diisobutyl fumarate, diisobutyl maleate, diheptyl fumarate, diheptyl maleate, bis (2-ethylhexyl) fumarate, bis (2-ethylhexyl) maleate, (−)-Dimenthyl fumarate, (−)-Bis ((S)-1-(ethoxycarbonyl)ethyl) fumarate, (−)-Bis ((S)-1-(ethoxycarbonyl)ethyl) maleate, Bis (2-trifluoroethyl) fumarate, Bis (2-trifluoroethyl) maleate, and mixtures thereof.

26. The method of claim 22, wherein the compound of Formula (I) comprises dimethyl fumarate (DMF).

27. The method of claim 22, further comprising administering to the subject a therapeutically effective regime of methylene blue.

28. (canceled)

29. (canceled)

30. The method of claim 22, wherein the compound of Formula (I) is administered systemically.

31. The method of claim 22, wherein the compound of Formula (I) is administered intravenously.

32. The method of claim 22, wherein the therapeutically effective regime comprises multiple administrations of the compound of Formula (I).

33. The method of claim 22, wherein the therapeutically effective regime comprises administration of the compound of Formula (I) at a dose in the range of from about 200 mg to about 800 mg per day.

34. The method of claim 22, wherein the therapeutically effective regime comprises administration of the compound of Formula (I) at a dose in the range of from about 480 mg to about 720 mg per day.

35. The method of claim 22, wherein the therapeutically effective regime comprises administration of methylene blue at a dose in the range of from about 0.25 mg/kg/hour to about 1.0 mg/kg/hour.

36. (canceled)

37. The method of claim 22, wherein the compound of Formula (I) is formulated as a nanoparticle.

38. The method of claim 22, wherein the compound of Formula (I) is formulated for controlled and/or sustained release.

39. The method of claim 22, wherein the compound of Formula (I) is formulated in a cyclodextrin.

40. The method of claim 39, wherein the cyclodextrin is selected from the group consisting of hydroxypropyl-β-cyclodextrin, endotoxin controlled β-cyclodextrin sulfobutyl ethers, or cyclodextrin sodium salts.

41. The method of claim 22, wherein the subject is a human.

42. The method of claim 22, wherein the subject has a muscle disorder or a liver disorder.

43. The method of claim 42, wherein the muscle disorder involves muscle wasting.

44. The method of claim 42, wherein the muscle disorder is selected from the group consisting of Cancer cachexia, age-related muscle wasting (sarcopenia), Mitochondrial myopathy, Acid Maltase Deficiency (AMD), Amyotrophic Lateral Sclerosis (ALS), Amyotrophy, Andersen-Tawil Syndrome, Anterior compartment syndrome of the lower leg, Becker Muscular Dystrophy (BMD), Becker Myotonia Congenita, Bethlem Myopathy, Bimagrumab, Bulbospinal Muscular Atrophy (Spinal-Bulbar Muscular Atrophy), Carnitine Deficiency, Carnitine Palmityl Transferase Deficiency (CPT Deficiency), Cataplexy, Central core disease of muscle, Centronuclear Myopathy, Charcot-Marie-Tooth Disease (CMT), Charley horse, Chronic fatigue syndrome, Chronic progressive external ophthalmoplegia, Congenital Muscular Dystrophy (CMD), Congenital Myasthenic Syndromes (CMS), Congenital Myotonic Dystrophy, Contracture, Cori Disease (Debrancher Enzyme Deficiency), Cramp, Cricopharyngeal spasm, Debrancher Enzyme Deficiency, Dejerine-Sottas Disease (DSD), Dermatomyositis (DM), Diastasis recti, Distal Muscular Dystrophy (DD), Distal spinal muscular atrophy type 2, Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Exercise therapy for idiopathic inflammatory myopathies, Exercise-associated muscle cramps, Exertional rhabdomyolysis, Facioscapulohumeral Muscular Dystrophy (FSH or FSHD), Fibrodysplasia ossificans progressive, Finnish (Tibial) Distal Myopathy, Forbes Disease (Debrancher Enzyme Deficiency), Fukuyama Congenital Muscular Dystrophy, Glycogen storage disease type XI, Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery-Dreifuss Muscular Dystrophy), Hereditary inclusion body myopathy and myositis, Hereditary Motor and Sensory Neuropathy (Charcot-Marie-Tooth Disease), Hyperthyroid Myopathy, Hypertonia, Hypothyroid Myopathy, Inclusion-Body Myositis (IBM) and myopathy, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Laminopathy, Late-onset mitochondrial myopathy, Limb-Girdle Muscular Dystrophy (LGMD), Lou Gehrig's Disease (Amyotrophic Lateral Sclerosis), Macrophagic myofasciitis, McArdle Disease (Phosphorylase Deficiency), Merosin-Deficient Congenital Muscular Dystrophy, Metabolic myopathy, Mitochondrial Myopathy, Miyoshi Distal Myopathy, Motor Neurone Disease, Muscle atrophy, Muscle fatigue, Muscle imbalance, Muscle weakness, Muscle-Eye-Brain Disease, Myasthenia Gravis (MG), Myoadenylate Deaminase Deficiency, Myofibrillar Myopathy, Myopathy, Myopathy, X-linked, with excessive autophagy, Myophosphorylase Deficiency, Myositis, Myositis ossificans, Myostatin-related muscle hypertrophy, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Orofacial myological disorders, Paramyotonia Congenita, Paratonia, Pearson Syndrome, Pelvic floor muscle disorder, Periodic Paralysis, Peroneal Muscular Atrophy (Charcot-Marie-Tooth Disease), Phosphofructokinase Deficiency, Phosphoglycerate Kinase Deficiency, Phosphorylase Deficiency, Polymyositis (PM), Pompe Disease (Acid Maltase Deficiency), Progressive External Ophthalmoplegia (PEO), Psoas muscle abscess, Pyomyositis, Rod Body Disease (Nemaline Myopathy), Sarcoglycanopathy, Sphincter paralysis, Spinal Muscular Atrophy (SMA), Spinal-Bulbar Muscular Atrophy (SBMA)/Kennedy's disease, Steinert Disease (Myotonic Muscular Dystrophy), Strain (injury), Tarui Disease (Phosphofructokinase Deficiency), Thomsen Disease (Myotonia Congenita), Thyrotoxic periodic paralysis, Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), ZASP-Related Myopathy and Zenker's degeneration.

45. The method of claim 42, wherein the muscle disorder is a muscular dystrophy.

46. The method of claim 42, wherein the liver disorder is selected from the group consisting of mitochondrial liver disease, hepatitis, alcoholic liver disease, fatty liver disease (hepatic steatosis), NASH-Non-alcoholic steatohepatitis, Gilbert's syndrome, cirrhosis, primary liver cancer, primary biliary cirrhosis, primary sclerosing cholangitis, and Budd-Chiari syndrome.

47. The method of claim 22, wherein the subject does not have a neurodegenerative disorder.

48. The method of claim 22, wherein the subject does not have multiple sclerosis (MS), Alzheimer's disease (AD), amyotrophic lateral sclerosis (ALS), Parkinson's disease (PD), Huntington's disease (HD), Mitochondrial myopathy or a progressive external ophthalmoplegia.


SEQUENCE LISTING (48 sequences)

SEQ ID NO: 1 - DNA, 20 bp, Artificial Sequence
  Description of Artificial Sequence Synthetic primer
cacccaagaacagggtttgt

SEQ ID NO: 2 - DNA, 20 bp, Artificial Sequence
  Description of Artificial Sequence Synthetic primer
tggccatgggtatgttgtta

SEQ ID NO: 3 - DNA, 25 bp, Artificial Sequence
  Description of Artificial Sequence Synthetic primer
tgctgtctccatgtttgatgtatct

SEQ ID NO: 4 - DNA, 22 bp, Artificial Sequence
  Description of Artificial Sequence Synthetic primer
tctctgctccccacctctaagt

SEQ ID NO: 5 - DNA, 21 bp, Artificial Sequence
  Description of Artificial Sequence Synthetic primer
gtgattcaccgcaggaaaagc

SEQ ID NO: 6 - DNA, 22 bp, Artificial Sequence
  Description of Artificial Sequence Synthetic primer
gtgcgacgtagaagatcctttc

SEQ ID NO: 7 - DNA, 20 bp, Artificial Sequence
  Description of Artificial Sequence Synthetic primer
aggaacacggagtgacccaa

SEQ ID NO: 8 - DNA, 21 bp, Artificial Sequence
  Description of Artificial Sequence Synthetic primer
tatgctcggtgtaagtagcca

SEQ ID NO: 9 - DNA, 20 bp, Artificial Sequence
  Description of Artificial Sequence Synthetic primer
caactactcccaggttgccc

SEQ ID NO: 10 - DNA, 21 bp, Artificial Sequence
  Description of Artificial Sequence Synthetic primer
agtgactgaaacgtagccgaa

SEQ ID NO: 11 - DNA, 20 bp, Artificial Sequence
  Description of Artificial Sequence Synthetic primer
tggtttgagcgagtgttcat

SEQ ID NO: 12 - DNA, 20 bp, Artificial Sequence
  Description of Artificial Sequence Synthetic primer
ccttcttactccggaagggt

SEQ ID NO: 13 - DNA, 20 bp, Artificial Sequence
  Description of Artificial Sequence Synthetic primer
acaggtatttccgggtggtc

SEQ ID NO: 14 - DNA, 19 bp, Artificial Sequence
  Description of Artificial Sequence Synthetic primer
cgccattctggatcggcat

SEQ ID NO: 15 - DNA, 21 bp, Artificial Sequence
  Description of Artificial Sequence Synthetic primer
catataccaaatctctccctc

SEQ ID NO: 16 - DNA, 21 bp, Artificial Sequence
  Description of Artificial Sequence Synthetic primer
gtgcgagatagtagtagggtc

SEQ ID NO: 17 - DNA, 18 bp, Artificial Sequence
  Description of Artificial Sequence Synthetic primer
gtaggattggtgctgtgg

SEQ ID NO: 18 - DNA, 18 bp, Artificial Sequence
  Description of Artificial Sequence Synthetic primer
ggatcctcccgaatcaac

SEQ ID NO: 19 - DNA, 20 bp, Artificial Sequence
  Description of Artificial Sequence Synthetic primer
tgccatccactacatgacgg

SEQ ID NO: 20 - DNA, 20 bp, Artificial Sequence
  Description of Artificial Sequence Synthetic primer
gctctgtccaccaaatgcac

SEQ ID NO: 21 - DNA, 20 bp, Artificial Sequence
  Description of Artificial Sequence Synthetic primer
tggggcctgcagttcttatg

SEQ ID NO: 22 - DNA, 20 bp, Artificial Sequence
  Description of Artificial Sequence Synthetic primer
atggtgtggcagcggtatag

SEQ ID NO: 23 - DNA, 20 bp, Artificial Sequence
  Description of Artificial Sequence Synthetic primer
accccctaggaatcacctcc

SEQ ID NO: 24 - DNA, 20 bp, Artificial Sequence
  Description of Artificial Sequence Synthetic primer
gcctaggaggtctggtgaga

SEQ ID NO: 25 - DNA, 20 bp, Artificial Sequence
  Description of Artificial Sequence Synthetic primer
gagcacgaccatcgaaaacg

SEQ ID NO: 26 - DNA, 20 bp, Artificial Sequence
  Description of Artificial Sequence Synthetic primer
cgatatgccagcttccgact

SEQ ID NO: 27 - DNA, 20 bp, Artificial Sequence
  Description of Artificial Sequence Synthetic primer
cgccgaccgttgactattct

SEQ ID NO: 28 - DNA, 20 bp, Artificial Sequence
  Description of Artificial Sequence Synthetic primer
cggctcgaataaggaggctt

SEQ ID NO: 29 - DNA, 20 bp, Artificial Sequence
  Description of Artificial Sequence Synthetic primer
acctttcatgatcacgccct

SEQ ID NO: 30 - DNA, 20 bp, Artificial Sequence
  Description of Artificial Sequence Synthetic primer
gggcaggatagttcagacgg

SEQ ID NO: 31 - DNA, 20 bp, Artificial Sequence
  Description of Artificial Sequence Synthetic primer
tgctcccattcatgctgagg

SEQ ID NO: 32 - DNA, 20 bp, Artificial Sequence
  Description of Artificial Sequence Synthetic primer
ctccagcaccaccaaaaagc

SEQ ID NO: 33 - DNA, 20 bp, Artificial Sequence
  Description of Artificial Sequence Synthetic primer
gaagcgccaccctagcaata

SEQ ID NO: 34 - DNA, 20 bp, Artificial Sequence
  Description of Artificial Sequence Synthetic primer
gcttggattaaggcgacagc

SEQ ID NO: 35 - DNA, 20 bp, Artificial Sequence
  Description of Artificial Sequence Synthetic primer
gccaacacagtgctgtctgg

SEQ ID NO: 36 - DNA, 22 bp, Artificial Sequence
  Description of Artificial Sequence Synthetic primer
ctgcttgctgatccacatctgc

SEQ ID NO: 37 - DNA, 20 bp, Artificial Sequence
  Description of Artificial Sequence Synthetic primer
tccgagcatcttatccacgc

SEQ ID NO: 38 - DNA, 20 bp, Artificial Sequence
  Description of Artificial Sequence Synthetic primer
gtatggtggtactcccgctg

SEQ ID NO: 39 - DNA, 20 bp, Artificial Sequence
  Description of Artificial Sequence Synthetic primer
atggtccacaatgagcccag

SEQ ID NO: 40 - DNA, 20 bp, Artificial Sequence
  Description of Artificial Sequence Synthetic primer
gaacgaatgactctgcccct

SEQ ID NO: 41 - DNA, 22 bp, Artificial Sequence
  Description of Artificial Sequence Synthetic primer
atacttcgtcacacaagcaaca

SEQ ID NO: 42 - DNA, 23 bp, Artificial Sequence
  Description of Artificial Sequence Synthetic primer
ggcctagttttatggatagggct

SEQ ID NO: 43 - DNA, 25 bp, Artificial Sequence
  Description of Artificial Sequence Synthetic primer
catctgttctgattctttgggcacc

SEQ ID NO: 44 - DNA, 25 bp, Artificial Sequence
  Description of Artificial Sequence Synthetic primer
tgggctcatacaataaagcctagaa

SEQ ID NO: 45 - DNA, 20 bp, Artificial Sequence
  Description of Artificial Sequence Synthetic primer
gcagtccggcttacagctaa

SEQ ID NO: 46 - DNA, 20 bp, Artificial Sequence
  Description of Artificial Sequence Synthetic primer
ggtagctgttggtgggctaa

SEQ ID NO: 47 - DNA, 20 bp, Artificial Sequence
  Description of Artificial Sequence Synthetic primer
ggctgtattcccctccatcg

SEQ ID NO: 48 - DNA, 22 bp, Artificial Sequence
  Description of Artificial Sequence Synthetic primer
ccagttggtaacaatgccatgt